(19) United States
(12) Patent Application Publication
(10) Pub. No.: US 2023/0079448 A1
(43) Pub. Date: Mar. 16, 2023

(54) DEVICES AND METHODS FOR NEGATIVE PRESSURE THERAPY

(71) Applicant: APPLIED TISSUE TECHNOLOGIES LLC, Hingham, MA (US)

(72) Inventor: Elof Eriksson, Hingham, MA (US)

(73) Assignee: APPLIED TISSUE TECHNOLOGIES LLC, Hingham, MA (US)

(21) Appl. No.: 17/790,432

(22) PCT Filed: Jan. 8, 2021
(86) PCT No.: PCT/US21/12739

§ 371 Date: Jun. 30, 2022

Related U.S. Application Data
(60) Provisional application No. 62/959,551, filed on Jan. 10, 2020.

(51) Int. Cl. A61M 1/00; A61F 13/14; A61F 13/02

(52) U.S. Cl. CPC A61M 1/917; A61F 13/14; A61F 13/0216

(57) ABSTRACT

Devices and methods for edema management, e.g., reducing edema, are disclosed. A treatment device may include a chamber defining a treatment space, the chamber being fabricated from a substantially impermeable material and configured to conform to the shape of the periphery, an inner surface of the chamber including a plurality of surface patterns, the chamber having a sealing portion at a base; and at least one conduit connected to the chamber and in fluid communication with the treatment space so as to enable the application of a negative pressure to the treatment space. The treatment device may be configured for reducing edema at a target site on a subject. The treatment device may be further configured for edema management, e.g., reducing edema, of at least one breast, e.g., both breasts, of a subject.

### FIELD OF THE TECHNOLOGY

The disclosure relates generally to edema management and target site healing and, more particularly, to devices and methods for reducing edema and promoting healing at target sites with negative pressure and/or therapeutic agents.

### BACKGROUND

Various techniques are employed to treat open wounds, such as incisions after a surgical procedure, and to reduce edema at various sites on the body. For example, open wounds may be treated with moist or dry gauze. Edema may be managed using compressive wraps, reducing the temperature at an affected site, elevation of the affected site above heart level, and pharmaceuticals, such as diuretics. However, such treatment may result in excessive pain, dehydration of the wound, loss of fluids and proteins, loss of heat or delayed healing. To delay the appearance of infection, burn wounds may be additionally treated with antibacterial creams and the like.

Devices for protecting treatment sites and providing environmental control of the treatment site have been developed. For example, exemplary devices and methods for use are described in U.S. Pat. No. 5,152,757, entitled “System for Diagnosis and Treatment of Wounds.” by Elof Eriksson, and U.S. patent application Ser. No. 11/130,490, entitled “Wound Chamber With Remote Access Portal,” by Eriksson et al., each of which is incorporated herein by reference as if set forth in its entirety. Also incorporated herein by reference in their entirety for all purposes are U.S. Pat. Nos. 8,298,197, 8,632,523, 9,693,908, and 9,717,829, all to Eriksson et al.

A device typically includes a chamber for enclosing a predetermined surface area about a site on a patient. The device is sealed to the skin immediately adjacent to the site. However, certain sites on and around a limb may not be treatable by a device that is intended for use on relatively flat skin surfaces. Instead, it may be necessary to enclose all or a portion of a limb in a chamber in order to create a chamber environment around the site. In addition to other features, the device may have a conduit for introducing treatment fluid and treatment additives into the device and extracting wound fluid and/or air from the device.

Many sites can be treated by the application of negative pressure. The method of such treatment has been practiced for many years. The benefits of such treatment can include: decreased bacterial burden; reduction of edema; reduction of exudate and interstitial fluids; reduction of wound size; promotion of blood flow and facilitation of circulation, and stimulation of formation of granulation tissue. On a cellular level, negative pressure wound therapy promotes pro-inflammatory gene expression which initiates migration and differentiation of cells involved in angiogenesis and the proliferative stages of wound healing. Existing devices and appliances for the provision of negative pressure therapy are complex. Such devices typically encompass a porous insert such as foam or gauze that is placed on the site; a conduit connecting the inner space to a source of suction; a flexible cover draped over these components and sealed to the skin around the site; an electrically powered suction pump; controls to operate the pump and monitor the system; containers to collect exudate; filters to process the materials removed from the site; and safety systems to prevent harm to the patient and to block the escape of biological materials into the outside environment. These devices are expensive, labor intensive, and restrictive of patient mobility. These devices are generally not considered suitable for sites on certain areas of the body, including the breasts, the face, the neck, and the head. The many components, particularly the seals around the insert and the conduit, tend to leak. Therefore, suction must be applied either continuously or frequently.

Continuous suction is typically achieved by a vacuum pump powered by an electric motor. Such systems require complex means to measure, monitor, and control the operation of the pump to ensure the safety of the patient. In addition, many negative pressure devices are contraindicated in the presence of necrotic tissue, invasive infection, active bleeding, and exposed blood vessels. They require the use of a porous insert (sponge, foam, gauze, mesh, etc.) in the wound. The insert may present two problems: growth of tissue into the insert, and the harboring of infectious and/or undesirable materials in the insert. Tissue can grow into and around such inserts, thereby causing adverse results to the healing process. Moreover, such inserts can retain exudates and microorganisms, and therefore can become contaminated and/or infected, presenting an adverse effect to the healing process. In addition, the high cost of these devices may deter or delay their use on patients.

Existing negative pressure treatment devices are labor intensive since they require the user to assemble, fit, and customize a number of components. First, the user must prepare, trim and size an insert of foam, gauze, mesh, or other material that will be placed on the target site. Next, the user must position a conduit in the insert, and then cover the conduit and insert with a material that is intended to create a leakproof seal. In practice, and as mentioned above, such compositions tend to leak, requiring the frequent application of suction in order to establish and re-establish negative pressure within the space about the wound. In addition, currently available negative pressure devices and systems block the view of the site, making monitoring and diagnosis more difficult.

### SUMMARY

In accordance with one or more embodiments, devices and methods for reducing edema at target sites using negative pressure and/or therapeutic agents are disclosed.

In accordance with one or more embodiments, a method of reducing edema at a target site on a subject may comprise securing a treatment device to a periphery of the target site; applying a negative pressure to a treatment space of the treatment device to allow a plurality of surface patterns to directly contact the target site and create pathways configured to evenly distribute the negative pressure in the treatment space; and maintaining the negative pressure in the treatment space for a duration of time sufficient to reduce edema of the target site. The treatment device may comprise a chamber that defines the treatment space, the chamber being fabricated from a substantially impermeable material and configured to conform to the shape of the periphery, an inner surface of the chamber including the plurality of surface patterns, the chamber having a sealing portion at a base; and at least one conduit connected to the chamber and in fluid communication with the treatment space so as to enable the application of a negative pressure to the treatment space.

In some embodiments, the target site comprises a wound site.

In some embodiments, the method includes reducing edema in a tissue surrounding the wound site, e.g., healthy tissue.

In some embodiments, the target site is associated with a breast of the subject. In some embodiments, the treatment device is sized and shaped to be placed on at least one breast of the subject. In certain embodiments, the plurality of surface patterns are shaped to contour to the at least one breast of the subject. In some embodiments, a treatment device is secured to both breasts of the subject.

In some embodiments, the plurality of surface patterns comprises a plurality of embossed structures.

In some embodiments, the target site is associated with a lower leg of the subject. In particular embodiments, the target site is associated with a foot of the subject. In some embodiments, the target site is associated with an arm of the subject. In particular embodiments, the target site is associated with a hand of the subject. In some embodiments, the target site is associated with a chest of the subject. In some embodiments, the target site is associated with an abdomen of the subject.

In some embodiments, the target site results from an elective procedure. In particular embodiments, the elective procedure is a cosmetic procedure. In some embodiments, the target site results from a surgical procedure. In particular embodiments, the surgical procedure produces an incision.

In particular embodiments, the target site results from a skin graft procedure.

In further embodiments, the method may comprise collecting exudate from the target site.

In further embodiments, the method may comprise debriding the target site prior to the securing of the treatment device.

In further embodiments, the method may comprise flushing the target site with a sterile solution.

In further embodiments, the method may comprise applying a therapeutic agent to the target site.

In accordance with one or more embodiments, an edema management device for reducing edema at a target site on a subject may comprise a chamber defining a treatment space, the chamber being fabricated from a substantially impermeable material and configured to conform to a shape of a periphery of the target site, an inner surface of the chamber including a plurality of surface patterns; a sealing portion at a base of the chamber configured to be secured to the skin of the periphery around the target site, the sealing portion comprising an adhesive; and at least one conduit having a first end and a second end, the first end connected to the chamber and in fluid communication with the treatment space so as to enable applying a negative pressure to the treatment space.

In some embodiments, the chamber is constructed and configured to enclose a target site on the subject experiencing edema.

In some embodiments, the at least one conduit is configured to remove fluid from, or introduce fluid to, the treatment space.

In some embodiments, the plurality of surface patterns are shaped to contact and contour to the target site. The plurality of surface patterns may be positioned at a distance of about 0.2 mm to about 10 mm apart from one another and may have a height of about 0.1 mm to about 5 mm. In some embodiments, the plurality of surface patterns is positioned in a uniform pattern on the inner surface of each chamber. In some embodiments, the plurality of surface patterns is positioned in a non-uniform pattern on the inner surface of each chamber.

In some embodiments, each of the plurality of surface patterns has a shape selected from the group consisting of a cone, a pyramid, a pentagon, a hexagon, a half sphere, a dome, a rod, an elongated ridge with round sides, and an elongated ridge with square sides.

In some embodiments, the plurality of surface patterns intrude into the isolated treatment space in a direction generally perpendicular to the inner surface of the chamber. In certain embodiments, the plurality of surface patterns comprises a plurality of embossed structures.

In further embodiments, the edema management device may comprise a suction device for applying the negative pressure, a fluid trap, and an exhaust port, wherein the suction device is in fluid communication with the treatment space of each chamber, the fluid trap and the exhaust port, and wherein the fluid trap is fluidly positioned between the suction device and the exhaust port.

In some embodiments, the target site is associated with the breast of the subject. In some embodiments, the target site is associated with a lower leg of the subject. In particular embodiments, the target site is associated with a foot of the subject. In some embodiments, the target site is associated with an arm of the subject. In particular embodiments, the target site is associated with a hand of the subject.

In accordance with one or more embodiments, an edema management device for at least one breast of a subject may comprise a chamber having an inner surface defining a treatment space sized and shaped to enclose at least one breast, the inner surface comprising a plurality of surface patterns configured to directly contact the at least one breast and to create pathways for distributing a negative pressure between the inner surface and the at least one breast; a sealing portion at a base of the chamber configured to be secured to the skin of the chest wall around the at least one breast, the sealing portion comprising an adhesive; and at least one conduit having a first end and a second end, the first end connected to the chamber and in fluid communication with the treatment space so as to enable applying a negative pressure to the treatment space.

In some embodiments, the chamber is sized and shaped to enclose both breasts of the subject.

In some embodiments, the chamber is configured to provide support to the at least one breast when a negative pressure is applied.

In some embodiments, the chamber is associated with an undergarment configured to provide support to the at least one breast. The undergarment may include an outer supporting structure, such as a pouch or pocket, that encloses the chamber. In particular embodiments, the outer supporting structure is a fabric material.

In some embodiments, the chamber is fabricated from a substantially impermeable material.

In some embodiments, the at least one conduit is configured to remove fluid from, or introduce fluid to, the treatment space.

In certain embodiments, the plurality of surface patterns are shaped to contour to the at least one breast. The plurality of surface patterns may be positioned at a distance of about 0.2 mm to about 10 mm apart from one another and may have a height of about 0.1 min to about 5 mm. In some embodiments, the plurality of surface patterns is positioned in a uniform pattern on the inner surface of each chamber. In some embodiments, the plurality of surface patterns is positioned in a non-uniform pattern on the inner surface of each chamber.

In some embodiments, each of the plurality of surface patterns has a shape selected from the group consisting of a cone, a pyramid, a pentagon, a hexagon, a half sphere, a dome, a rod, an elongated ridge with round sides, and an elongated ridge with square sides.

In some embodiments, the plurality of surface patterns intrude into the isolated treatment space in a direction generally perpendicular to the inner surface of the chamber.

In particular embodiments, the chamber does not include a porous insert.

In further embodiments, the edema management device may comprise a suction device for applying the negative pressure, a fluid trap, and an exhaust port, wherein the suction device is in fluid communication with the treatment space of each chamber, the fluid trap and the exhaust port, and wherein the fluid trap is fluidly positioned between the suction device and the exhaust port.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention and are not intended as a definition of the limits of the invention. For purposes of clarity, not every component may be labeled in every drawing. In the following description, various embodiments of the present invention are described with reference to the following drawings, in which:

FIG. 1 is a perspective view of a treatment device with a conduit leading from a chamber to a suction source;

FIG. 2 is a side sectional view of the device in FIG. 1;

FIG. 3 is a sectional view of the device in FIG. 1 with an additional conduit leading to a port;

FIG. 4 is a sectional view of the device in FIG. 1 with a branching conduit leading to a port;

FIG. 5 is a perspective view of the end of the conduit communicating with the inner chamber space;

FIG. 6 is a side sectional view of a plurality of surface patterns disposed on and into the inner surface of the chamber wall, where the plurality of surface patterns are of uniform size and shape, and are spaced uniformly apart;

FIGS. 6a, 6b, and 6c present schematics of a plurality of surface patterns in accordance with one or more embodiments;

FIG. 7 is a side sectional view of two groups of surface patterns disposed on and into the inner surface of the chamber wall, where one group intrudes into the treatment space, the other group intrudes to a lesser extent, and surface patterns from these groups alternate in a regular pattern;

FIG. 8 is a side sectional view of three groups of surface patterns disposed on and into the inner surface of the chamber wall, where such groups have varying degrees of intrusion into the chamber space and alternate in a regular pattern;

FIG. 9a is an overview of surface patterns disposed on and into the inner surface of the chamber wall, where the structures consist of raised ridges;

FIG. 9b is a side sectional view of the raised ridges of FIG. 9a with rounded edges;

FIG. 9c is a side sectional view of the raised ridges of FIG. 9a with square cross sections;

FIG. 10 is an overview of the raised ridge surface patterns shown in FIG. 9a, with the addition of raised dome surface patterns positioned among the ridges;

FIG. 11 is an overview of raised ridge surface patterns disposed on and into the inner surface of the chamber wall, where two parallel lines of such surface patterns form a channel;

FIG. 12 is an overview of raised dome surface patterns disposed on and into the inner surface of the chamber wall, where two parallel lines of such surface patterns form a channel;

FIG. 13 is a view of a chamber, showing a pattern of channels leading to the center of the chamber and then to the conduit communicating from the inner of the treatment space;

FIG. 14 is a view of a radiating pattern of channels leading to the communicating conduit;

FIG. 15 is a view of a branching pattern of channels leading to the communicating conduit;

FIG. 16 is a view of a sub-branching pattern of channels leading to the communicating conduit;

FIG. 17 is a side sectional view of a fold in the chamber wall;

FIG. 18a is a side sectional view of a fold in the chamber wall, with a plurality of surface patterns disposed on and into the inner surface of the fold, with the surface patterns maintaining a continuous open space within the fold;

FIG. 18b is a side sectional view of the fold in the chamber wall of FIG. 17 with a plurality of surface patterns disposed on the inner surface of the fold;

FIG. 19 is a view of a chamber configured as a conduit for placement over a limb, and having a plurality of surface patterns and channels on the inner surface of the chamber wall;

FIG. 20 is a sectional view of the device in FIG. 1 showing a fluid collector placed before the suction source;

FIG. 21 is a sectional view of a suction device in the form of a squeeze bulb of deformable material;

FIG. 22 is a sectional view of a suction device in the form of a flexible chamber containing one or more compression springs;

FIG. 23 is a sectional view of a suction device in the form of a wedge-shaped chamber containing one or more torsional springs;

FIG. 24 is a sectional view of the device in FIG. 23 containing a flat spring;

FIG. 25 is a sectional view of a suction device with a trap and filter incorporated into the exhaust port;

FIG. 26 is a perspective view of a treatment device for wounds on the face, head, and neck in accordance with one or more embodiments;

FIG. 27 is a perspective view of another embodiment of a treatment device for treatment sites associated with the face, head, and neck in accordance with one or more embodiments;

FIG. 28 is a perspective view of an unassembled treatment device for treatment sites associated with the face, head, and neck in accordance with one or more embodiments;

FIG. 29 is a front view of an assembled treatment device for treatment sites associated with the face, head, and neck in accordance with one or more embodiments;

FIG. 30 is a perspective view of an exudate collection device in accordance with one or more embodiments;

FIG. 31 is a schematic side view of an exudate collection device in accordance with one or more embodiments;

FIG. 32 is an exploded schematic side view of an exudate collection device in accordance with one or more embodiments;

FIG. 33a is a perspective view of a treatment device in accordance with one or more embodiments;

FIG. 33b is a side view of a chamber treatment device in accordance with one or more embodiments;

FIGS. 34a-34d and 35a-35d present data discussed in the accompanying Examples;

FIGS. 36a-36b show front (FIG. 36a) and side (FIG. 36b) views of a treatment device for attachment to the breasts of a subject in accordance with one or more embodiments;

FIG. 37 illustrates treatment devices of the present disclosure of different dimensions;

FIG. 38 illustrates a pie chart of the surgical procedures performed on a subject population prior to treatment using a treatment device in accordance with one or more embodiments;

FIGS. 39a-39d illustrate treatment of a median sternotomy incision at different stages using a treatment device of this disclosure. FIG. 39a illustrates the closed incision prior to dressing application, i.e., day 0. FIG. 39b illustrates the closed incision following dressing application and maintenance of −80 mm Hg pressure on day 0. FIG. 39c illustrates the closed incision immediately following dressing removal on day 4. FIG. 39d illustrates the closed incision's appearance at day 13;

FIGS. 40a-40d illustrate treatment of a thoracotomy incision at different stages using a treatment device of this disclosure. FIG. 40a illustrates the closed incision prior to dressing application, i.e., day 0. FIG. 40b illustrates the closed incision following dressing application and maintenance of −80 mm Hg pressure on day 0. FIG. 40c illustrates the closed incision immediately following dressing removal on day 3. FIG. 40d illustrates the closed incision's appearance at day 9;

FIGS. 41a-41d illustrate treatment of a laparotomy incision at different stages using a treatment device of this disclosure. FIG. 41a illustrates the closed incision prior to dressing application, i.e., day 0. FIG. 41b illustrates the closed incision following dressing application and maintenance of −80 mm Hg pressure on day 0. FIG. 41c illustrates the closed incision immediately following dressing removal on day 3. FIG. 41d illustrates the closed incision's appearance at day 14;

FIGS. 42a-42d illustrate treatment of an incision from a breast reconstruction procedure at different stages using a treatment device of this disclosure. FIG. 42a illustrates the closed incision prior to dressing application, i.e., day 0. FIG. 42b illustrates the closed incision following dressing application and maintenance of −80 mm Hg pressure on day 0. FIG. 42c illustrates the closed incision immediately following dressing removal on day 5. FIG. 42d illustrates the closed incision's appearance at day 14; and

FIGS. 43a-43d illustrate treatment of an incision from a full-thickness skin graft (FTSC) donor site at the patient's groin at different stages using a treatment device of this disclosure. FIG. 43a illustrates the closed incision prior to dressing application, i.e., day 0. FIG. 43b illustrates the closed incision following dressing application and maintenance of −80 mm Hg pressure on day 0. FIG. 43c illustrates the closed incision immediately following dressing removal on day 4. FIG. 43d illustrates the closed incision's appearance at day 13.

While the invention is susceptible to various modifications and alternative forms, specific embodiments thereof have been shown by way of example in the drawings and ae described below in detail. It should be understood, however, that the description of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION

The present disclosure is directed to providing a simple, safe, disposable, and cost-effective edema management device that is easy to install and operate, that allows freedom of motion to the patient, and that overcomes, or at least reduces the effects of, one or more of the problems set forth above. In at least some embodiments, the present disclosure does not require the use of an interface layer such as a porous insert. In some embodiments, a one-piece, two-piece, or multi-piece construction of the device is suitable for patient treatment and eliminates virtually all leaks, therefore preserving and maintaining negative pressure at the target site without the need for constant or frequent regeneration of negative pressure. In addition, the structure of the device is configured to reduce edema, promote target site healing, and to create pathways through which negative pressure can be distributed and maintained in the treatment space. The device may contact the target site directly, without the use of an interface layer such as a porous insert. In some embodiments, the device may be used in conjunction with a therapeutic agent, such as an immediate release or sustained-release therapeutic agent. The therapeutic agents may be formulated as described further herein, such as with a biomaterial. The biomaterial may be naturally occurring and biocompatible. In some embodiments, the biomaterial may be cross-linked with the therapeutic agent. The biomaterial may be selected based on its properties. In some embodiments, the biomaterial may be, for example, a hydrogel, a hydrocolloid, alginate, or any other gel. Devices of the invention may be made of a substantially impermeable material such as to promote healing. e.g., by reducing water loss, and to facilitate the use of therapeutic agents. In at least some embodiments, the permeability of the material may generally relate to a moisture and/or water vapor transmission rate. An exudate collection device may interface with and be used in conjunction with the treatment device. The indications for the present disclosure may be expanded beyond the limitations imposed on current devices. The cost-effectiveness of the present disclosure may lead to the provision of negative pressure therapy on a more widespread basis and earlier in the timeline of target site care.

In accordance with various aspects and embodiments, the devices and methods of the present disclosure may be used to treat non-procedural edema, e.g., diabetic joint swelling or swelling during pregnancy, lymphedema, e.g., swelling in the arms, legs, or both due to a lymphatic system or other blockage, full and partial thickness burns, traumatic wounds, post-surgical wounds from elective, e.g., cosmetic procedures, or non-elective procedures, sores and ulcers, e.g., diabetic ulcers, infected wounds, post-infection wounds, skin loss due to dermatological conditions, and other conditions that result in skin and deep tissue loss. In some non-limiting embodiments, a patient may have a total body surface area (TBSA) injury of about 5% to about 30% or greater. In some embodiments, the target site comprises a wound site. In some embodiments, the wound site may be a bilateral wound. The wounds may be acute or chronic. In at least some embodiments, deep dermis, subcutaneous tissue, muscle, fascia, tendon or bone may be exposed due to the nature of the target site. The devices and methods may be used anywhere on the human body and may also be suitable for veterinary applications. For example, treatment sites that are suitable for use with devices of the invention include, but are not limited to, treatment sites associated with the breasts, the lower leg, e.g., foot, the arm, e.g., hand, the chest, and the abdomen. As a non-limiting example, devices and methods of the invention may be useful in treating an edematous lower leg even without a preceding surgical procedure, such as a result from a traumatic injury, e.g., a contusion, or complications due to diabetes or pregnancy.

The devices and methods may, for example, be used for target site preparation to prevent scarring as well as the fixation and protection of skin grafts during treatment. In some specific non-limiting embodiments discussed herein, treatment may relate to injuries occurring on the head, face, and/or neck. For example, a patient's cheeks, forehead, and/or crown may be treated. The head including facial tissue presents unique challenges which may be addressed in accordance with one or more embodiments. In other specific non-limiting embodiments discussed herein, edema and incision management may occur at or near at least one breast, such as after a cosmetic procedure or a surgical procedure, such as a mastectomy. Incision and skin management after surgery on the breast is often complicated by poor circulation of the skin and delayed healing of incisions in combination with marked swelling of the entire breast. This often results in prominent scarring and often deformation of the shape of the breast. In some non-limiting embodiments, the edema management device may be placed on a wound site resulting from a surgical procedure, such as a sternotomy, thoracotomy, laparotomy, or the donor site of a full-thickness skin graft.

One aspect of the present disclosure is seen in an edema management device for reducing edema at a target site on a subject including a chamber defining a treatment space around the target site. The chamber may be formed from a flexible, substantially moisture and gas impermeable material and be configured to conform to a shape of a periphery of the target site. The sealing portion at the base of the chamber forms a water-tight and gas-tight seal using a suitable adhesive material. At least one conduit communicates from the treatment space to a source of suction. The at least one conduit may be configured to remove fluid from, or introduce fluid to, the treatment space. In at least some embodiments described herein, the source of suction is capable of generating and/or maintaining sub-atmospheric pressure within an enclosed space. The suction source also serves as a receptacle for materials removed from the chamber, including exudates from a target site, such as drained fluids and debris. All components preferably are inexpensive, lightweight, and disposable.

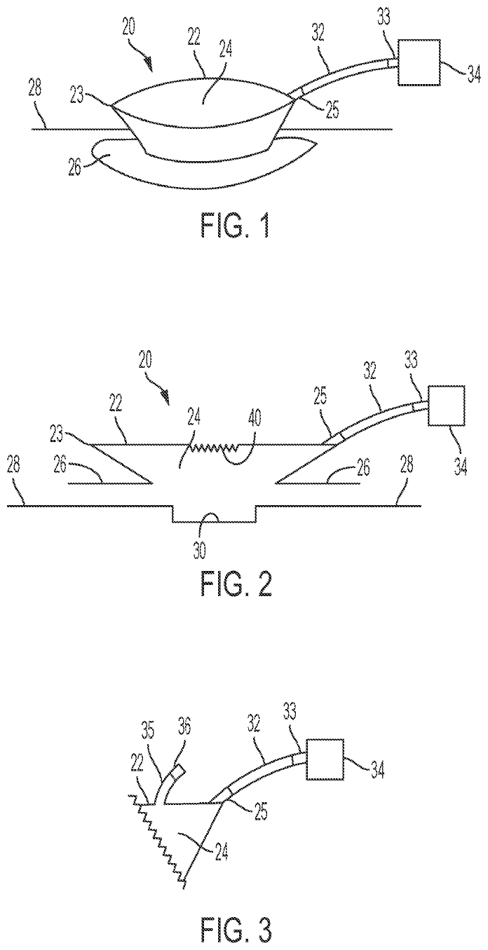

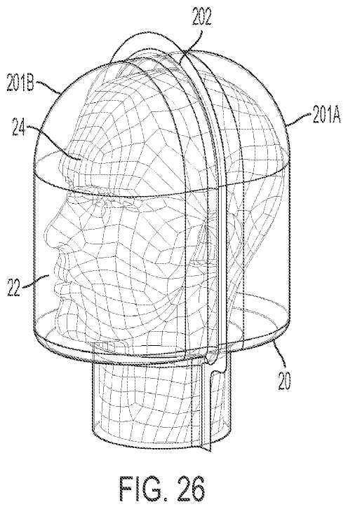

Referring to FIGS. 1 and 2, views of a treatment device 20 are provided. The device 20 includes a chamber 22 defining a treatment space 24 and a base 26 that may be sealed to a skin surface 28 of a patient over a target site 30. In the illustrated embodiment, the chamber 22 has a bellows configuration with a fold 23. However, the invention is not so limited, and other configurations of a chamber formed of a flexible, substantially moisture and gas impermeable material may be used. The chamber may also be transparent to allow for visual inspection of the target site during treatment. In accordance with certain embodiments, the material is substantially transparent. In other embodiments, the material may be substantially opaque. The use of a substantially impermeable material is particularly advantageous for introducing therapeutic agents to the target site. Additionally, the impermeable nature of the chamber material prevents water loss from the target site, which facilitates improved healing. Materials from which the device 20 may be made will be discussed in further detail below. The device 20 can be designed for use with any target site on any body part, including both human and veterinary applications. Various geometries such as circular, square, rectangular, tubular, pouch, envelope or other shapes may be implemented based on the intended application. In accordance with some embodiments, chamber 22 defining treatment space 24 may be configured for a specific body part. For example, a chamber in the form of a tube or sleeve for placement over a limb is shown in FIG. 19, whereas a chamber in the form of a hood for placement over a head is shown in FIG. 26 as described further below.

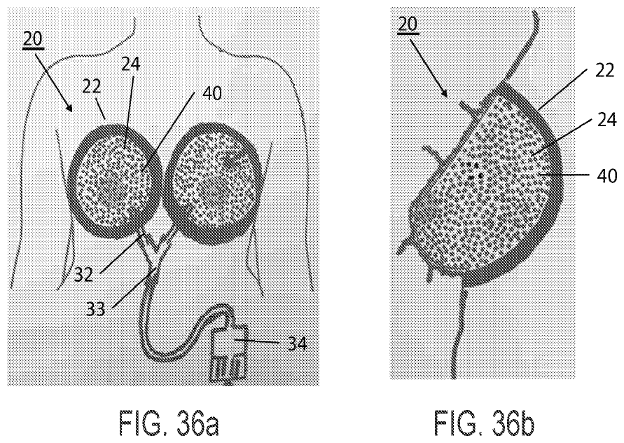

In some cases, a treatment device may have a chamber shaped for placement onto at least one breast as shown in FIGS. 36a and 36b. The treatment device may be unitary. e.g., a single device that encloses both breasts, or may be two separate devices that each cover a single breast as shown in FIG. 36a. Independent of the number of treatment devices utilized for the breast, the chamber of the treatment device has an inner surface that defines a treatment space sized and shaped to enclose the at least one breast, a scaling portion at the base of the treatment device that configured to be secured to the skin of the chest wall around the at least one breast using an adhesive, and at least one conduit having a first end and a second end. The first end of the conduit may be connected to the chamber and in fluid communication with the treatment space so as to enable applying a negative pressure to the treatment space. In some cases, in a device that has two chambers, the conduits from each chamber may be used independently; alternatively, the conduits from each chamber may be fluidly connected to have a single flow path.

The treatment device for the at least one breast may be configured to provide support to the breasts. For example, the treatment device, when adhered to the breast(s) and depressurized, may have sufficient rigidity to support the breast(s) when worn with standard clothing, such as a shirt or similar. Alternatively, or in addition, the treatment device and any chambers thereof may be associated with an undergarment that is configured to provide support to the breast(s), such as brassiere or similar undergarment. The treatment device may be directly incorporated into an undergarment having a series of straps with releasable fasteners, such as snaps or hook-and-loop, to secure the treatment device around the chest and/or shoulders and to the breast(s). In some cases, the treatment device may be associated with an outer supporting structure of an undergarment, such as a pocket or pouch, that is sized and shaped to accept the treatment device. In this configuration, the undergarment comprising the outer supporting structure includes closures typically found on undergarments, such as straps with releasable fasteners or the like. The outer supporting structure, and the undergarment overall, may be made from any suitable fabric material that is used for undergarments; suitable fabric materials for undergarments would be known by those skilled in the art.

In accordance with one or more embodiments, the geometry of the treatment device chamber may generally involve a degree of convexity or concavity in order to enable it to be pulled down, for example, into contact with a deep or full thickness wound, such as upon application of negative pressure. In some non-limiting embodiments, the material used to form the chamber and/or its shape may impart this design in terms of conforming to anatomical and/or target site contours. For example, the material used to form the chamber may have a convexity that substantially matches the general shape of a breast and may be sized in accordance with one of the standard cup sizes for breasts (i.e., A. B. C, D, etc.). In some embodiments, the device may be a concave structure that is generally hallowed inward towards the target site. A major redundancy (concavity with respect to the device) of the construction is highly desirable in certain cases such as deep wounds. For example, in a deep wound such as a puncture, the underside of a generally concave device may be pulled into every deep part of the wound in order to provide negative pressure wound therapy and micromechanical forces to the entire deep wound surface. An additional reason for redundancy in the construction of the device is to provide folds that extend into the deep part of the target site, thus creating channels for air and fluid flow.

Still referring to FIGS. 1 and 2, a dermal or cutaneous adhesive material may be provided on a sealing portion of the base 26 for providing a fluid-tight seal with sufficient adhesive strength to prevent inadvertent removal of the chamber 22 or breach of the fluid-tight seal during normal patient movement. Numerous adhesive materials sufficient for these purposes are known to those of ordinary skill in the art.

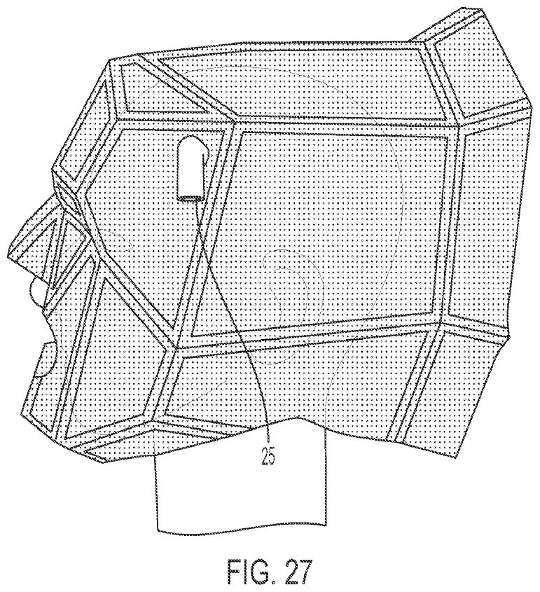

In accordance with some embodiments, device 20 can be specifically designed for treating target sites associated with the head, face, and/or neck. The device may cover only the head, or both the head and neck. Referring again to FIG. 26, a chamber may be formed that fits over the head and attaches at the base of the neck for example at, or proximate to, the collar bone. In some embodiments, the chamber may be defined by a single piece of material, or a two or more-piece design may be implemented. The device may be oversized to fit any size head or may be customized. Oversized devices may lead to the presence of wrinkles in use which may be advantageous for negative pressure distribution. In accordance with other embodiments and referring to FIG. 26, the device may be comprised of two pieces 201A and 201B that may adhesively join to form a seam 202, for example, around the midline of the head and across the ears. In some embodiments, the pieces may be joined by other methods, and may for example, by a zipper type seal. Still, in accordance with other embodiments and as shown in FIG. 27, the device may be made from a plurality of pieces that contour the face.

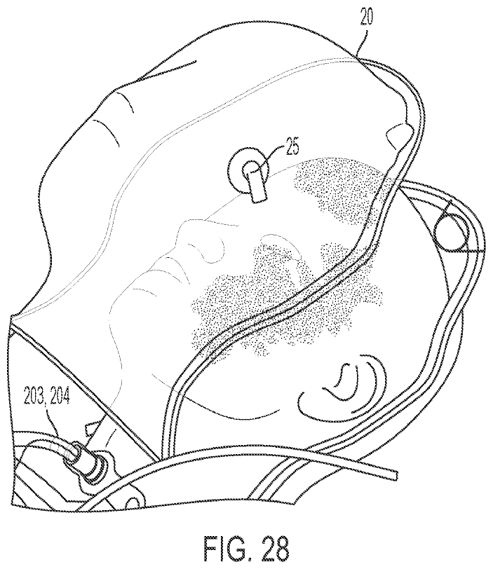

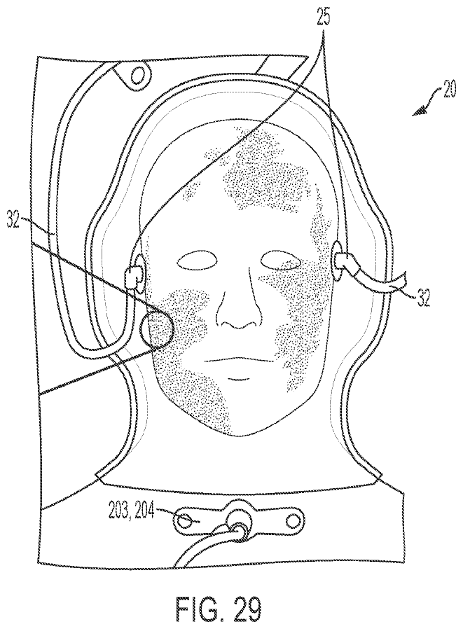

The head device may or may not have openings for the nose and mouth. If the device is constructed to cover the nose and mouth, the nose and mouth may be surgically sealed for treatment. For example, soft conforming obturators, or alternatively, sutures may be used. Referring to FIG. 28, a patient being treated with a device 20 covering the nose and mouth may breathe via an installed tracheostomy tube 203 and be fed via an installed gastrostomy tube 204. These tubes may be fitted outside of the device, for example, at the neck. Contact lenses (not shown), such as oversized lenses, may be used to protect the eyeballs during treatment.

A conduit 32 having a first (or proximal) end and a second (or distal) end is attached to the chamber 22 preferably at a location spaced above the base 26 and communicates with the treatment space 24. The conduit 32 is constructed to maintain its shape without collapsing and to permit the passage of exudate, such as drainage fluids and debris. The conduit 32 may be permanently fixed to the chamber 22, or a fitting, such as a tubular port 25 may be provided to allow the attachment and removal of the conduit 32 or any other device that can deliver material or therapeutic agents to, or remove material from, the treatment space 24. The conduit 32 may terminate at a wall of the chamber 22, or it may extend through the wall a distance and terminate within the treatment space 24, where it may communicate with such space, with channels formed on the inner surface of the chamber wall, or with folds formed in the chamber wall. The conduit 32 is sealed to the chamber 22 in such a manner as to prevent the escape of liquid or gas from the treatment space 24 to the outside environment. The distal or second end of the conduit 32 terminates at a device that generates sub-atmospheric pressure, such as suction device 34. The suction device 34 may be a pump, although other types of devices may be used as discussed below. A fitting 33 may be provided to permit the detachment and reattachment of a suction device 34 to the conduit 32. Referring to FIG. 36a, a treatment device 20 that incorporates two chambers 22, one for each breast, each chamber 22 may have a conduit 32 that is fluidly connected to a fitting 33 that merges their individual fluid paths into one, such as a Y-connector or similar, for connection to a suction device 34.

Referring to FIG. 28, when device 20 is used to treat face, head, and/or neck wounds, the device 20 may comprise a plurality of tubular ports 25 for communication with one or more tubes 32. The ports 25 may be strategically placed based on the geometry of the chamber. At least one tubular port 25 may be located at the top of the device and at least one tubular port 25 may be located at the bottom of the device. In accordance with some embodiments, tubular ports may be positioned on the side of the patient's face, at the temples proximate the ears. In some embodiments, there are at least two tubular ports present.

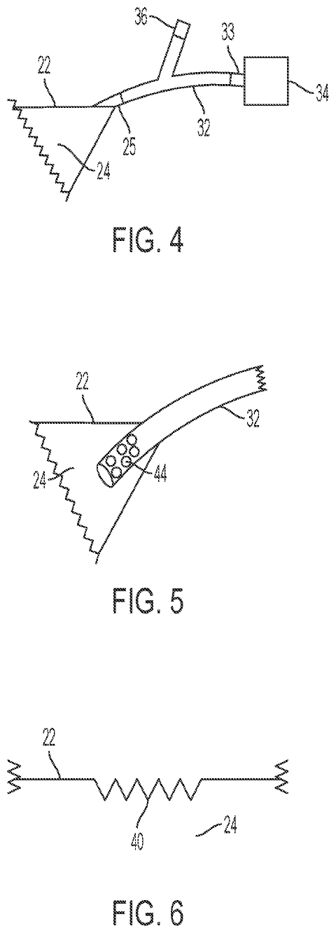

Turning to FIG. 3, a sectional view of the device 20 is provided, showing a second conduit 35 attached to the chamber 22 and communicating with the treatment space 24, with channels, or with folds. A distal end of the conduit 35 terminates in a portal 36. The disclosure is not limited to any number of communicating conduits, and multiple conduits and portals may be provided for accessing the treatment space 24. FIG. 4 shows the device in FIG. 1 with a branch of the conduit 32 that leads to a portal 36. The portal 36 may be used for the delivery of therapeutic agents-such as antimicrobials, antibiotics, antifungals, and analgesics-prior to, during, or after the delivery of negative pressure. As such, the portal 36 may be a lure configured for attaching to a container or a syringe. Alternatively, therapeutic agents may be delivered through the same conduit 32 that communicates with the suction device 34.

In accordance with some embodiments, the treatment device may allow for the delivery of a therapeutic agent directly to the target site. As such, the therapeutic agents can be delivered in concentrations significantly higher than could otherwise be administered intravenously or orally. For example, antibiotics can be applied directly to the target site at concentrations in a range from about the conventional oral concentration to up to about 1000 times the conventional oral concentration (i.e., 1000×MIC), or even higher. If ingested or administered directly to the bloodstream, these concentrations would be toxic to the body. Topical application facilitates the use of significantly higher concentrations that facilitate healing. Combinations of therapeutic agents, such as analgesics, antibiotics, and chemical or enzymatic debriding agents may also be used, and may advantageously reduce or eliminate the need for surgical debridement of the target site. The therapeutic agents may be delivered in concentrations of 1 times MIC to at least 1,000 times MIC. In some embodiments, concentrations of up to 5.000 times MIC during shorter periods of treatment may be used. For larger target site, for example, a limiting factor may be systemic toxicity from absorption from the target site. Because of the combination of half-life absorption and surface area, the total amount of pharmacologic agent should generally be limited to 5 times a standard total IV dose in a 24-hour period. In some non-limiting embodiments concentrations of 1,000 to 5000×MIC may be safely and effectively administered. In other non-limiting embodiments. 15 to 500 to 1000×MIC concentrations may be safely and effectively administered and may find particular utility, for example, in various military applications. Concentration, volume, absorption rate, and surface area may all be considerations and defined variables in terms of targeted and accurate delivery of various therapeutic agents in accordance with various embodiments. In various embodiments, a treatment device as described herein may be used to form a reservoir of a formulated therapeutic agent to promote availability for healing. Use of the devices in combination with formulated therapeutic agents described herein may demonstrate a synergistic effect in terms of efficiency in target site healing and edema management.

In accordance with one or more embodiments, one or more therapeutic agents may be formulated and delivered for improved efficacy and/or usability in connection with a treatment device. Analgesics and/or antibiotics and/or anti-fungals and/or debriding chemicals and/or anti-inflammatory agents and/or scar-reducing agents and/or chemotherapeutic agents and/or sterile fluids may be delivered to a target site, whether small molecules or macromolecules. In some embodiments, therapeutic agents may be formulated for sustained-release to enable prolonged care. Maintenance of desired placement of therapeutic agents relative to a target site may also be promoted by their formulation as described herein. High-dose topical treatment of target sites, not possible systemically, may be enabled via delivery of active treatment agents in liquid or gel form to the device. In some embodiments, an antibiotic may be gentamicin, vancomycin, clindamycin, minocycline, or tetracycline. In some embodiments, an anti-fungal may be DIFLUCAN® or amphotericin. In some embodiments, an analgesic may be lidocaine. In some embodiments, an anti-inflammatory agent may be a steroid or a non-steroidal anti-inflammatory, such as indomethacin or aspirin. In some embodiments, a scar-reducing agent may be cortisone. In some embodiments, a chemotherapeutic agent may be fluorouracil (5FU). One or more of these and other various therapeutic agents may be implemented alone or in combination.

In some embodiments, one or more therapeutic agents or combination thereof may be formulated with a gel for delivery to a target site. As discussed, herein, a hydrogel, hydrocolloid, alginate, or any other gel may be used. The formulation can be formed and then delivered to the treatment chamber. Alternatively, a gel may be positioned within the treatment chamber prior to the delivery of one or more therapeutic agents. Sustained-release delivery of one or more therapeutic agents from a reservoir of a gel may be provided. A layer of gel, such as a layer which is 2 to 10 mm thick, may be applied. In one embodiment, a 3 mm thick layer of gel may be used, and the total amount of gel may be 100 mL (100 cc). When using, for instance, vancomycin or gentamicin, the MIC is typically around 2 micrograms per mL or cm³ for common bacteria. A total of 200 mg of each of these antibiotics may be required in 100 mL of gel. For purposes of example only, an upper arm which has a surface area of approximately 2,000 cm² would use 60 cm³ of gel based on a 3 mm thick layer.

A hydrogel is typically a three-dimensionally cross-linked network composed of hydrophilic polymers with high water content. A hydrogel, gel, hydrocolloid, alginate, methyl cellulose, gelatin or any other gel may impart sustained-release characteristics to the therapeutic agent and may also promote the staying in place of the therapeutic agent relative to the target site being treated. For example, the gel may allow the therapeutic agent to be released for at least 24 hours, for at least 48 hours, for at least 72 hours, or for at least 96 hours. The half-life of active antibiotics may be approximately 24 hours, 48 hours, 96 hours, or longer in connection with embodiments involving sustained release formulations of a gel and/or antibiotic and/or analgesic. The drug release kinetics may be controlled by diffusion of the drug through the network. The polymer concentration and molecular weight, and type and extent of chemical and physical crosslinking allow for manipulation of the physical properties of the gel, such as viscoelasticity and drug release kinetics. Alginate, for example, may be used in various formulations of therapeutic agent(s) to achieve an acceptable viscosity range. Relatively high viscosity may be a desirable property to facilitate treatment, as well as relatively low freezing point.

The gel may be selected for at least one of its properties. For example, the gel may be selected for its pre-gel rheological properties, mechanical stability post-gelation, or control over the release of the incorporated therapeutic agent. Prior to gelation, the gel precursor may have sufficiently low viscosity to allow it to flow into molds and conform to the skin-facing side of the device. Subsequent to gelation, the gel must possess appropriate stiffness to maintain its form and not allow leakage from the device if it is ruptured, and must possess sufficient toughness to prevent its fracture with handling of the device.

According to Table 1, gel-therapeutic agent preparations comprising an antibiotic and a 0.5% (agarose) hydrogel may be stable for up to one week. Drugs dissolve passively into the gel and stay stable over time. In some embodiments, the gel-therapeutic agent preparation may further comprise saline. The gel may be biocompatible, meaning that it is structurally similar to the extracellular matrix in tissues. The gel may be flowable, allowing, for example, casting into a mold or injection via needle through a port. The loading of the hydrogen-therapeutic agent preparation in the device may allow consistent and uniform contact with the skin even with patient movement, maintaining continuous delivery of the therapeutic agent to the underlying tissue. The gel-therapeutic agent preparation may prevent loss of the therapeutic agent in the event of a minor leak in the adhesive of the sealing portion of the device.

TABLE 1
Activity of gel-based therapeutics after short-term storage.

| | |
|---|---|
| Original | Drug activity |
| concentration | (μg/mL) |

| Antibiotic | (μg/mL) | 3 days | 7 days |
|---|---|---|---|
| Gentamicin | 2.5 | 2.41 | 2.04 |
| Minocycline | 9.0 | 8.86 | 8.53 |

In some specific non-limiting embodiments, the gel may comprise a naturally derived polysaccharide. In some embodiments, the gel may comprise alginate, agarose, a hydrocolloid, cellulose or a combination thereof. In at least some embodiments, the gel may be a hydrogel.

In at least some embodiments, both an antibiotic and analgesic to reduce or eradicate infection and to ease pain may be implemented.

In some embodiments, the gel may be lyophilized prior to or subsequent to loading in the device to minimize weight, and facilitate storage, stability, and transport.

In accordance with one or more embodiments, the engineered skin-facing surface of the device chamber (having a patterned surface as described further below) may promote even distribution of a gel formulated therapeutic agents. Devices as described herein may also facilitate slow or sustained release of therapeutic agent.

In accordance with one or more embodiments, treatment devices may provide a sterile enclosure that can be applied immediately after injury to provide a protective dressing and a tool for precise topical delivery of therapeutic agents. The device can also serve as an incubator for strategic tissue regeneration in a controlled environment. The device is therefore capable of addressing problems of depth progression, infection, and sepsis from first treatment throughout the treatment process in both military and civilian populations. Prolonged field care and/or evacuations can beneficially be safely and comfortably endured until an advanced medical facility is reached. Rapid decontamination, reduced edema, reduced pain, reduced inflammation, reduced tissue loss, less scarring, and improved quality of healing may all be promoted.

In some embodiments, the device may be applied to a fresh target site within 24 hours of injury. In accordance with one or more embodiments, the device may remain in place for up to about four to seven days or longer before being replaced or removed entirely. Exudate from the target site may be removed and therapeutic agent a gel may be added to the device via a port. A transparent device may allow for target site evaluation. A sleeve may be placed over the device for additional protection. The device may be suitable for single-use sterile packaging for disposable one-time use. The gel may be rehydrated after storage.

In accordance with one or more embodiments, combination and/or serial treatment regimens may be practiced. The devices may be applied after hemostasis. A target site may be debrided for a period of time, and then subjected to treatment with the devices and formulated therapeutic agents described herein. In some non-limiting embodiments, the disclosed therapeutic agents may be left in place on the target site for up to a week or more prior to further evaluating the target site to discern further treatment steps. Beneficially, the target site may be left in the sterile environment of the disclosed devices for extended periods of time to facilitate healing. Furthermore, the formulated therapeutic agents also promote relatively uninterrupted periods of target site therapy. In some embodiments, periods of negative pressure target site therapy may be applied to the target site via the disclosed devices as described herein. Such therapy may be continuous or periodic, and may be performed simultaneously, prior to, or subsequent to application of the formulated therapeutic agents. In at least some embodiments, negative pressure therapy may be used sequentially with drug delivery. In certain embodiments, negative pressure therapy is not administered in parallel with drug delivery as may be an approach distinct from, for example, conventional target site irrigation techniques.

Turning now to FIG. 5, the end of the conduit 32 extending into the chamber space 24 is shown with multiple holes 44. The purpose of the holes 44 is to ensure that gases, liquids, fluid, debris, and other materials can flow and move out of the treatment space 24 into the conduit 32 without impediment.

The target site may advantageously be monitored through the substantially transparent chamber material. The negative pressure device 20 may also be equipped with sensors to monitor certain parameters within the chamber space 24. For example, oxygen, carbon dioxide, pH, temperature, and other parameters may be measured and monitored.

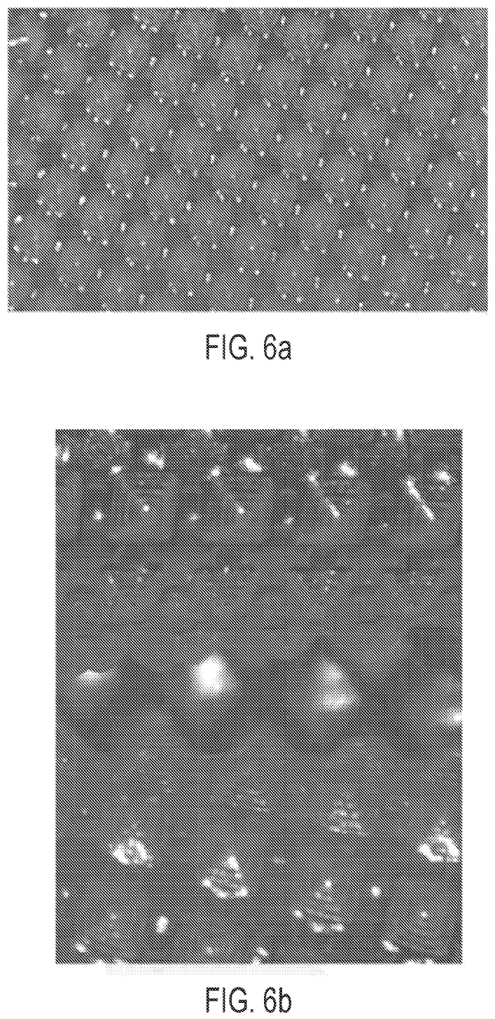

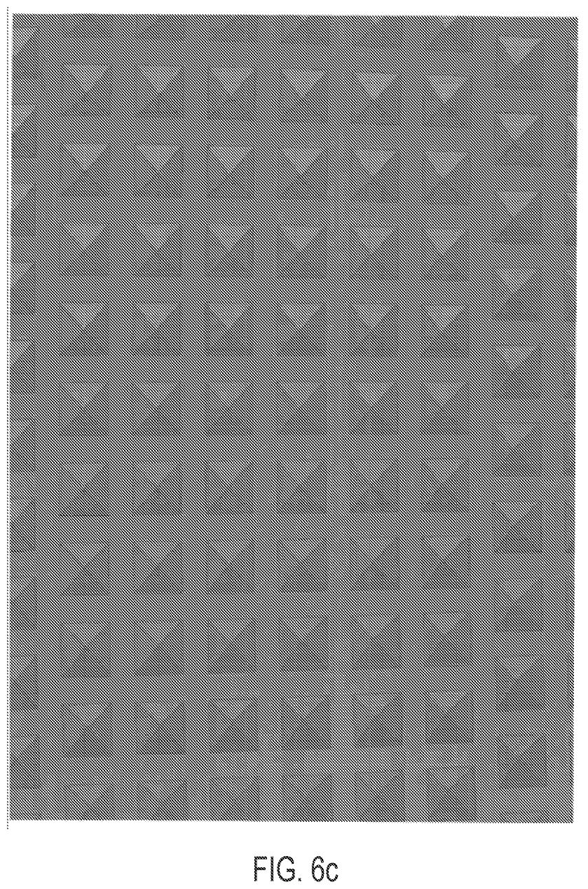

Referring to FIG. 6, the inner surfaces of the chamber wall may be configured with a plurality of surface patterns 40 that are engineered on the surfaces. FIGS. 6a-6c present schematics of different engineered surface patterns in accordance with one or more non-limiting embodiments. The portions of the inner surfaces with engineered surface patterns 40 may be varied from that shown in the figures, and preferably a high percentage of the inner surfaces include engineered surface patterns 40. The surface patterns preferably cover at least 50% of the inner surfaces, and more preferably at least about 95% of the inner surfaces. The plurality of surface patterns are raised when viewed from within the treatment space 24, and they intrude into such space in directions generally perpendicular to the inner surfaces of the treatment space 24. The plurality of surface patterns can be any shape, including without limitation a cone, a pyramid, a pentagon, a hexagon, a half sphere, a dome, a rod, an elongated ridge with rounded sides, or an elongated ridge with square sides. The plurality of surface patterns can be provided as identical shapes, or in any combination of shapes. The plurality of surface patterns can be provided with identical sizes, or in any combination of different sizes. The plurality of surface patterns can be provided in a regular or irregular pattern on the surface. The distance of intrusion into the treatment space 24 from the chamber wall by such surface patterns (height of such structures) is preferably between 0.01 mm and 20 mm, preferably between 1 mm and 10 mm, and most preferably about 2 mm. The spacing between such surface patterns is preferably between 0.01 mm and 5 mm, and the spacing for example, is most preferably about 2 mm apart. In some embodiments, about 2 mm high structures are arranged about 2 mm apart. When larger surface patterns are used, the structures may be spaced further apart and when smaller structures are used, the structures may be spaced closer together. For example, a configuration of pyramids of 0.2 mm in height may be spaced about 0.2 mm apart, whereas a configuration of larger pyramids of about 5 mm high may be spaced 10 mm apart.

The plurality of surface patterns 40 contact and contour to the target site during use of the device 20. The plurality of surface patterns may directly contact the target site surface. One purpose of the plurality of surface patterns is to ensure that negative pressure established within the treatment space 24 is evenly distributed and maintained throughout said space. As negative pressure is established within the conduit that leads to the source of suction or sub-atmospheric pressure, the chamber will lie tighter against the tissue of the target site. The device 20 includes the plurality of surface patterns 40 in order to define pathways to establish, distribute, and maintain negative pressure across the target site surface and prevent complete contact between the inner surfaces of the chamber and the target site tissue. Without such surface patterns, the chamber wall would make complete contact with the target site surface. As a result, there would be no space within which negative pressure could be established, distributed, and maintained. Therefore, the plurality of surface patterns are preferably semi-rigid. The term “semi-rigid” should be understood as meaning that deformation only occurs at a microscopic level under operating negative pressures in the range of 0.5-2 psi. Alternatively, the plurality of surface patterns may be somewhat flexible depending on the spacing between the individual features of the surface pattern. In addition, the plurality of surface patterns are designed to reduce the extent to which target site tissue can enter the space between the structures, so that a sufficient amount of open space is maintained. The plurality of surface patterns may be strategically patterned on the surface to produce desired negative pressure pathways within the chamber, and the shape and patterning of the inner surface of the chamber may be modified for a particular tissue type to maximize this effect.

An additional purpose of these structures is to serve as a form of stimulation to the target site to produce beneficial results, including without limitation, the formation of granulation tissue and an increase of micromechanical forces. Such micromechanical forces provide stimulation to a portion of the target site tissue, which has been suggested as a contributing factor to the effectiveness of negative pressure therapy. From the above discussion and the figures, it should be understood that the flexible chamber is movable over a range of positions around a target site of the periphery thereof. The range of positions includes a first position, such as the position shown in FIGS. 1 and 2, in which the plurality of surface patterns 40 are spaced apart from the opening of the chamber defined by the base 26. The range of positions also includes a second position in which at least some of the plurality of surface patterns 40 are positioned in the opening of the chamber. The second position is preferably a position in which the plurality of surface patterns 40 engage the target site.

The chamber wall can be formed of any appropriate medical grade material that has the following characteristics: flexibility, conformability, gas impermeability, liquid impermeability, the ability to be formed, tooled, and engineered, and the ability to retain the shape, function, and effectiveness of raised structures under desired ranges of negative pressure. The material should generally deter adhesion and in-growth. The material is preferably transparent to allow visual inspection of the target site during treatment. In addition, the material is preferably hypo-allergenic and provided to a medical facility in a sterile condition. For example, the chamber device may be made of a flexible, conformable material such as polyurethane, polyethylene, or silicone, although other similar materials may also be used. The material may have a thickness in the range of about 5 mm to about 100 mm. In some embodiments, the material may have a thickness of from about 1 mil up to about 100 mil. In some specific embodiments, a 5 mil polyurethane membrane may be used to form the treatment chamber.

The chamber is preferably designed to provide sufficient material to lie against the surface of the target site tissue without special sizing, trimming, or other customizing operations. The chamber may be made from a single ply of material, or may be constructed of multiple layers of material in and on which the plurality of surface patterns are engineered. It should be understood that a single ply chamber may be made of multiple sheets of material during manufacturing, but is provided to a medical facility in a state in which the multiple sheets are bonded or otherwise connected to one another. For example, individual three-dimensional shapes may be adhered or bonded to the inner surface of the chamber wall during manufacturing to provide the plurality of surface patterns. A single ply chamber could also be formed from a single sheet of material that defines both the chamber walls and the plurality of surface patterns. For example, the plurality of surface patterns may be embossed. Alternatively, a multiple layer chamber is provided to a medical facility in a state in which layers of material are stacked to form the chamber. For example, the layer facing the interior treatment space of the chamber could be a layer containing a plurality of surface patterns that is bonded onto a generally flat layer of material (or multiple sheets of generally flat layers) by a medical practitioner.

The plurality of surface patterns can be made by techniques familiar to those in the art, such as embossing, stamping, molding, forming, or bonding. If the structures are created by embossing their shape into the material, the embossed surface patterns may be left in a concave state relative to the outside of the chamber as shown in FIG. 6. Embossed surface patterns may also be formed on a single ply of material that also forms the walls of the chamber and the base. This may provide a chamber that is relatively flexible with semi-rigid structures on a single ply of material. Alternatively, the cavities may be filled with a suitable material to render the surface patterns solid. As another alternative, solid structures can be affixed to the inner surfaces of the chamber.

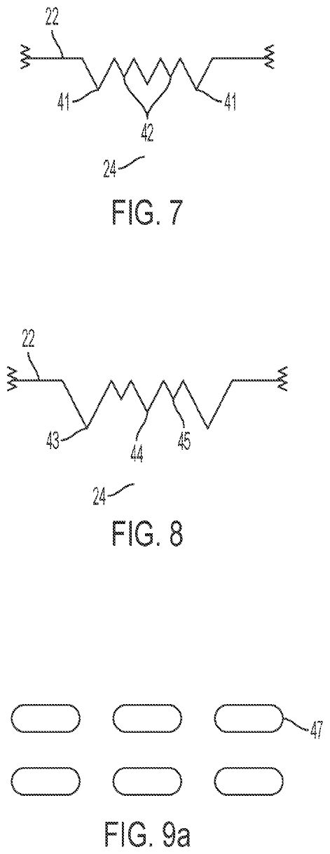

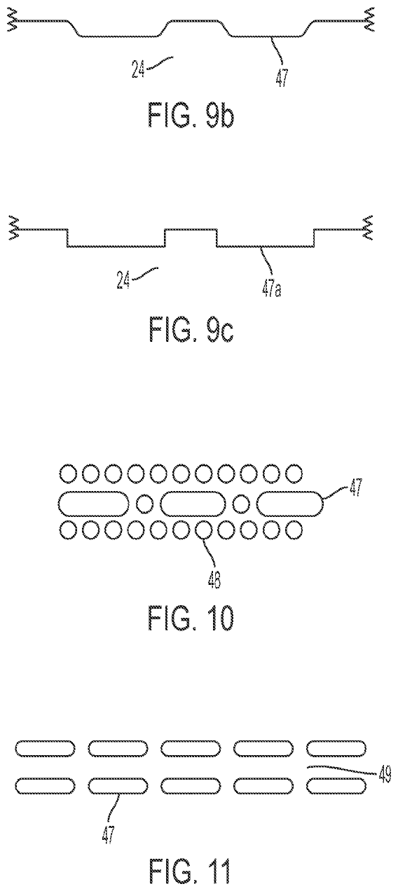

The raised structures on the inner surfaces of the chamber wall can be configured and distributed in a number of patterns. For example, FIG. 6 is a side sectional view of a portion of a chamber wall, showing a plurality of surface patterns 40 on the interior surface of the material that faces treatment space 24. The plurality of surface patterns 40 are identical in shape and size, and are positioned uniformly apart from one another. As another example. FIG. 7 is a side sectional view showing plurality of surface patterns 41 and 42 intruding into the chamber space, where surface patterns 41 intrude farther than surface patterns 42, and the plurality of surface patterns are configured in a regular alternating pattern of 41-42-41-42 and so forth. As yet another example. FIG. 8 is a side sectional view showing a plurality of surface patterns 43, 44, and 45 intruding into the treatment space, where surface patterns 43 intrude farther than surface patterns 44 and 45, surface patterns 44 intrude less than surface patterns 43 but farther than surface patterns 45, and surface patterns 45 intrude less than surface patterns 43 and 44. These surface patterns are configured in a regular alternating pattern of 43-45-44-45-43-45-44-45-43 and so forth. The embodiment shown in FIG. 8 makes it difficult for soft target site tissue to penetrate all of the spaces among the plurality of surface patterns. A sufficient amount of continuous space is established to make possible the distribution of negative pressure, as well as the addition of fluids, such as sterile liquids to flush the target site, fluids that debride the target site, or therapeutic agents, and the removal of exudate from the target site. As yet another example, FIG. 9a is an overview of a portion of the chamber wall, showing a plurality of surface patterns 47 in the form of raised ridges. The plurality of surface patterns 47 may be rounded (FIG. 9b), square (FIG. 9c), or a combination thereof when viewed from the side. As yet another example. FIG. 10 is an overview showing a plurality of surface patterns as a plurality of dome structures 48 interspersed with ridge structures 47. The plurality of dome structures 48 are preferably semi-spherical when viewed from the side, although other shapes are contemplated.

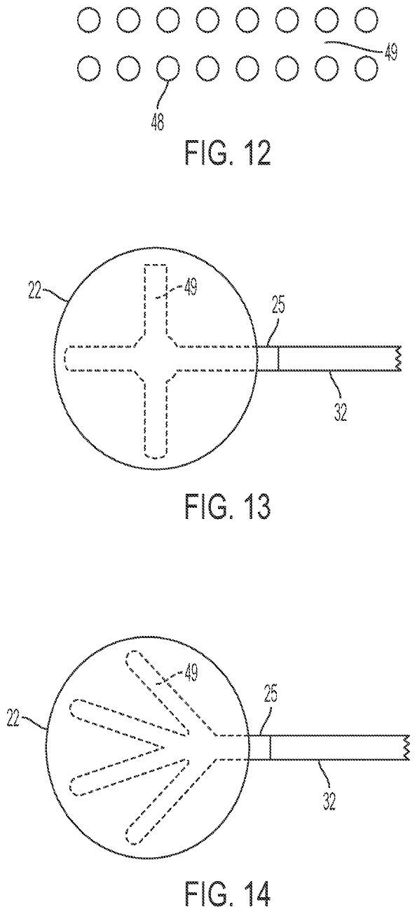

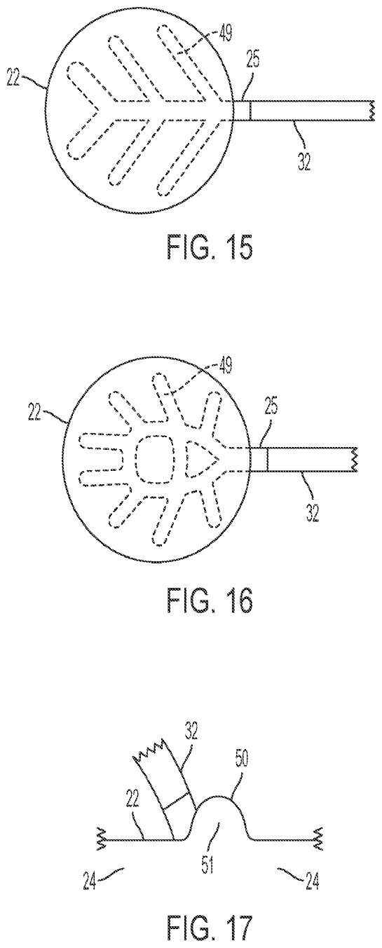

The distribution and maintenance of negative pressure within the chamber device and at all points on the target site may be enhanced by providing defined channel spaces as pathways among the plurality of surface patterns for the distribution of negative pressure. However, defined channel spaces are not required for providing fluid pathways within the treatment space. FIG. 11 is an overview of a portion of the chamber wall, showing a plurality of surface patterns 47 arranged in two parallel lines to form a channel 49. FIG. 12 shows a channel 49 formed by two parallel lines of raised domed structures 48. Such channels can be configured in various patterns, such as radial, circular, concentric, or branching. FIGS. 13-16 show overviews of patterns of channels 49 leading from tube 32 along the interior surface of chamber 22 facing treatment space 24. For each pattern, the channel 49 defines a space that opens directly to the treatment space 24. The space preferably opens to the treatment space 24 over the entire length of the channel 49.

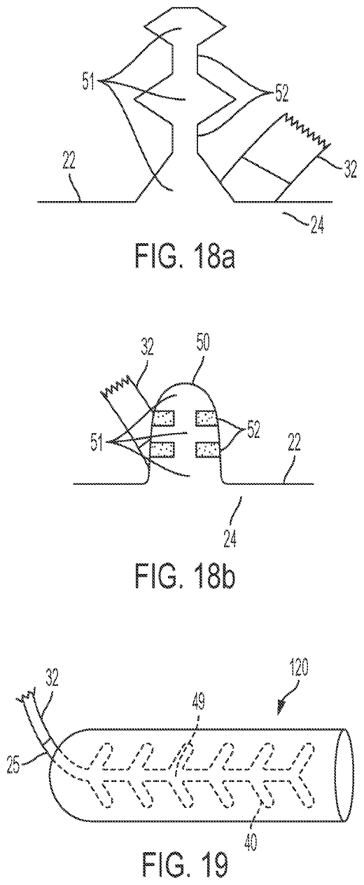

The distribution and maintenance of negative pressure within the chamber device and at all points on the target site can also be enhanced by the use of folds in the chamber wall to create additional channel space for the distribution of negative pressure. When negative pressure is established within the chamber, the material will tend to fold along the pre-formed location. FIG. 17 shows a channel 50 formed in a fold of the chamber wall. The channel 50 defines a space that opens directly to the treatment space 24. The space preferably opens to the treatment space 24 over the entire length of the channel 50. In order to increase the amount of channel space within such fold, the walls of the fold can be configured with a plurality of surface patterns that prevent the collapse of such space, and ensure continuous open space for the distribution and maintenance of negative pressure, and the passage of liquid, gas, and other material. As an alternative, FIG. 18a shows a plurality of surface patterns 52 that prevent the total collapse of the fold, and ensure continuous channel space 51. All channel spaces created on the interior surface of the chamber wall or by means of folds function as means to increase the effectiveness of distributing and maintaining negative pressure within the chamber, and also as means to enhance the effectiveness of removing gas, liquid, fluid, debris, and other materials from the chamber treatment space. As another alternative. FIG. 18b shows an embodiment similar to the embodiment shown in FIG. 17 with the addition of a plurality of surface patterns 52 on opposite sides of the fold. The plurality of surface patterns 52 are provided so that the fold will not collapse to the point where all of its interior surfaces form a tight seal against the movement of negative pressure. However, some of the interior surfaces, such as those adjacent to the fold, preferably contact the target site to provide stimulation as discussed above. The folds described in the previous embodiments are preferably formed at certain defined areas by molding or embossing the surfaces of the chamber 22.

FIG. 19 shows a treatment device 120 for delivering negative pressure and therapeutic substances in the form of a cylinder that can be placed over a limb. The treatment device 120 includes an open end and a closed end, though the chamber may have other shapes to accommodate other body parts, and may for example, be suitable for fitting over the head. The open end is preferably sealed with a cuff or collar (not shown), and the open end may include adhesive on the interior surface that defined a sealing portion. The treatment device 120 includes a plurality of surface patterns 40 and channels 49 on the interior surface of the chamber wall.

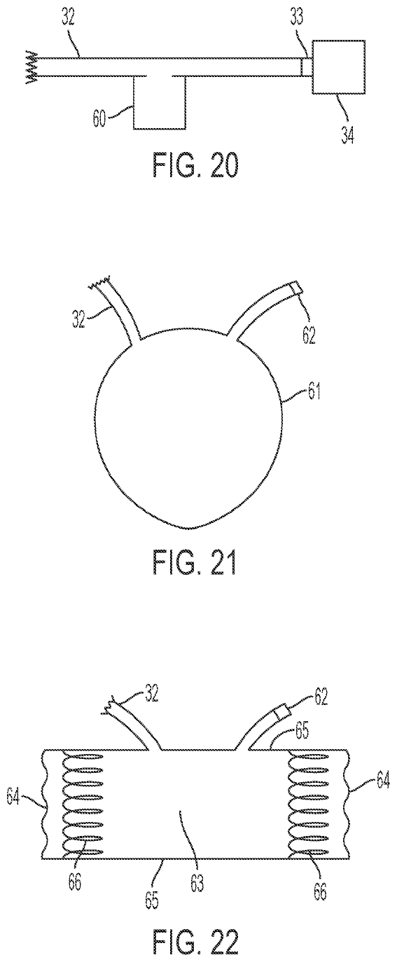

As shown in FIG. 20, a fluid collector 60 may be positioned on the conduit 32 between the chamber 22 and the suction device 34. The collector 60 is intended to receive exudates, such as fluids, extracted from the chamber space 24 and debris or material from the target site and store such materials for eventual disposal. The collector 60 may be detachable from the conduit 32, in order to replace a full collector with an empty collector.

Suction for the treatment device is provided by a suction device 34, which may be a pump that is connected and disconnected to the chamber device by appropriate connectors to provide sub-atmospheric pressure. Although the chamber can be used with a motor driven pump, it is also effective with a hand-powered device actuated by the caregiver or patient. The hand-powered device may be a squeeze bulb that provides suction by means of the energy stored in the material of its construction. Alternatively, the suction device may be powered by springs that are compressed by the user. The springs can be selected to produce the clinically desired level of negative pressure. The amount of suction provided by these suction devices is therefore dependent on the level of force generated by squeezed material or the springs. Unlike a motor driven suction pump, the hand powered device preferably cannot produce a high level of suction that may cause an adverse effect to healing.

Referring to FIG. 21, a suction device 61 in the form of a bulb constructed of a deformable material that stores the energy of deformation may be used. The conduit 32 communicates with the interior of the suction device 61. A one-way exhaust valve 62 also communicates with the interior of the suction device 61. When the user squeezes the suction device 61, air within the device is expelled through the exhaust valve 62. A portion of the energy used to deform the suction device 61 is stored in the material of which it is constructed, thus maintaining suction within the device, as well as within the conduit 32 and the chamber space 24. The bulb is selected and engineered to maintain a constant force and to maintain the clinically desired level of negative pressure within chamber space 24. Fluid from the target site 30 can flow through the conduit 32 into the suction device 61 where it can be stored prior to disposal. Once the suction device is full of fluid, the production of negative pressure ceases. The fluid capacity of the suction device thus operates as a safety shut-off mechanism without the need for electronic sensors and controls.

FIG. 22 shows an alternative suction device 63, consisting of flexible sides 64 and rigid sides 65. Compression springs 66 are located within suction device 63. The conduit 32 and the exhaust valve 62 both communicate with the interior of the suction device 63. When the user squeezes the rigid sides 65 towards one another, the springs 66 are compressed and air within the device is expelled through a one-way exhaust valve 62 thus maintaining suction within the device, as well as within the conduit 32 and the chamber space 24. The springs 66 are selected and engineered to maintain a constant force against rigid sides 65, and to maintain the clinically desired level of negative pressure within chamber space 24. Fluid from the target site 30 can flow through the conduit 32 into the suction device 63 where it can be stored prior to disposal of the entire device 63. This suction device will also cease operating when it is filled with fluid.

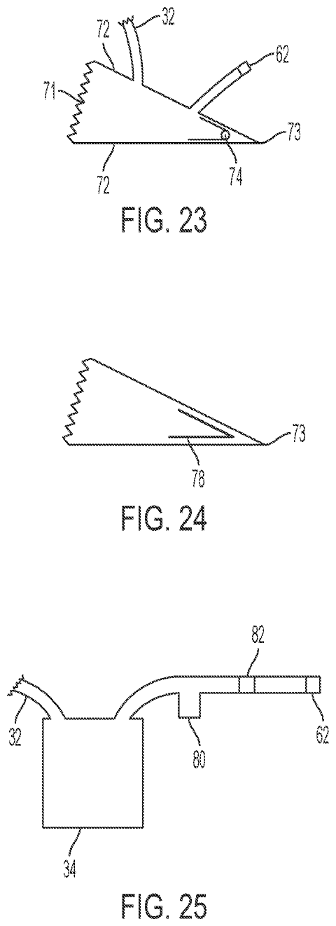

FIG. 23 shows an alternative suction device 70, consisting of rigid sides 72, joined by hinge 73, and flexible side 71. A torsional spring 74 is attached to either the interior or the exterior of rigid sides 72. The conduit 32 and the exhaust valve 62 both communicate with the interior of the suction device 70. When the user squeezes the rigid sides 72 towards one another, the spring 74 is compressed and air within the device is expelled through a one-way exhaust valve 62, thus maintaining negative pressure within the device, as well as within the conduit 32 and the chamber space 24. The spring 74 is selected and made to maintain a force against rigid sides 72 to maintain the clinically desired level of negative pressure within chamber space 24. Fluid from the target site 30 can flow through the conduit 32 into the suction device 70 where it can be stored prior to disposal of the entire device. FIG. 24 shows the device of FIG. 27 where the torsional spring 74 has been replaced by a flat spring 78.

For the previous suction devices, once suction has been established, fluid may flow from the target to the suction device, where it may be collected and stored for eventual disposal. Alternatively, a separate fluid collector, such as the fluid collector 60 in FIG. 20, can be positioned between the chamber and the suction device. Once the suction device has expanded to its original shape, suction ceases. The suction device will not continue to operate, and can be disconnected and disposed of. If treatment is to be continued, a new suction device can be connected and activated.

FIG. 25 is a sectional view of a trap 80 and a filter 82 interposed between the suction device 34 and the exhaust valve 62 for the purpose of preventing the expulsion of liquids or aerosols from the suction device.

The present disclosure can be engineered to operate at various levels of negative pressure, in accordance with clinical procedures. Traditional negative pressure devices can apply a negative pressure of between −0.5 and −2 psi, or about −750 mm Hg to about −125 mm Hg. The device of the present disclosure operates efficiently in this range. The chamber material conforms to the shape of the target site and any periphery thereof, and the plurality of surface patterns maintain their shape and functionality. However, the chamber can be engineered to operate at higher levels of negative pressure. The device of the present disclosure may also work efficiently at lesser negative pressures of, for example, from about −125 mm Hg to about −10 mm Hg. The application of less negative pressure may reduce pain and other complications. In addition, if a hand-powered suction device is used, the operating pressure of the device may be higher than the commonly accepted range; that is, the device may operate at a pressure close to 0 psi before suction ceases.

In accordance with one or more embodiments, a treatment device may be vacuum-assisted to facilitate circulation, e.g., stimulate blood flow and new blood vessel growth, biomechanically stimulate cells to encourage division and proliferation, and to remove factors that might inhibit healing such as bacteria. In further embodiments, a treatment device may be vacuum-assisted to reduce swelling due to a blockage in the lymphatic system, e.g., lymphedema, or a blockage in another portion of the circulatory system. e.g., a venous insufficiency. For example, application of negative pressure may aid in restoring proper drainage in the lymphatic system or a portion thereof, e.g., lymph nodes. Depending on the stage of treatment, connecting conduits can plug into different devices to apply negative pressure, flush or debride a target site, drain a target site, and deliver therapeutic agents to the target site.

The present disclosure eliminates many of the drawbacks to existing negative pressure therapy systems for edema and incision management. For example, the device of the present disclosure is preferably simplified and lightweight, and allows visual inspection of the target site and edema. In some embodiments of the disclosure, the patient is not restricted to a source of electricity or a battery pack. The system can be worn with ease, so that the patient's mobility is not otherwise compromised. In addition, the treatment device can be applied quickly without the need for custom fitting and construction. The device preferably does not leak due to the smooth adhesive scaling portion at the base, eliminating the need for constant suction from an electric pump with sophisticated controls and safety measure. There is no interface material such as a porous insert that can potentially cause tissue in-growth and harbor infectious material. Instead, the inner surfaces of the chamber are generally non-porous and non-adherent to prevent any interaction with the target site tissue. Further still, the suction pump preferably has built-in safety limitations on force of suction, duration of operation, and overfilling of the collector for fluid. The plurality of surface patterns may stimulate healing tissue at the target site and create an efficient pathway for the distribution of negative pressure. The chamber of the treatment device may be customized for use on any body part including limbs, head, face, or neck, and the breasts. The devices and methods disclosed herein may be used in conjunction with conventional tissue debridement and grafting techniques without any contraindications. The devices and methods may facilitate the fixation and protection of skin grafts and micrografts. The devices and methods may be superior to conventional approaches to administering negative pressure in terms of at least granulation tissue formation and edema management.

In accordance with one or more embodiments, a treatment device may be used in conjunction with a fluid collection device. A treatment system may therefore include a treatment device and a fluid collection device. The collection device may include an absorbable material, such as an absorbent insert. Any absorbable material, preferably biocompatible in terms of disposability, readily known to those of ordinary skill in the art may be implemented. The absorbable material may at least partially fill a compartment defined by the fluid collection device. The collection device insert may absorb fluid and package it for disposal during operation. All components preferably are inexpensive, lightweight, and disposable. In at least some embodiments, the fluid collection device may operate substantially as a filter or water trap.

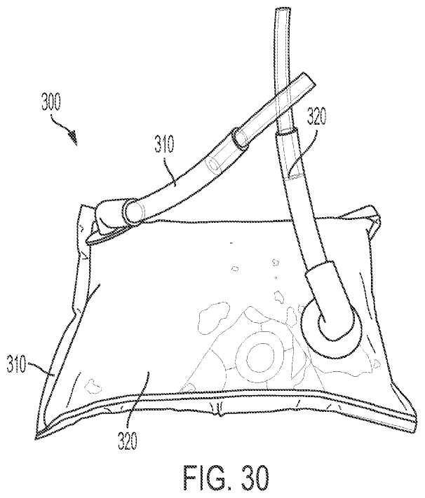

Referring to FIG. 30, a view of a fluid collection device 300 is provided. The device is intended to receive fluid, debris, exudate, or other materials removed from a target site during treatment or therapy and store such materials for eventual disposal. The device includes a compartment 310 defining a collection space. In the illustrated embodiment, the compartment has a pillow-shaped configuration. However, the invention is not so limited, and other configurations of a compartment formed of a flexible, substantially moisture and gas impermeable material may be used. The use of an impermeable material is particularly advantageous for preventing fluid loss from the compartment. Materials from which the device may be made vary. In some non-limiting embodiments, the collection device chamber may be made of the same material as the treatment device chamber.

The device can be designed for use with any treatment device as described herein and on any body part, including both human and veterinary applications. Various geometries such as spherical, cubic, parallelepiped, tubular, pouch, envelope or other shapes may be implemented based on the intended application.

In some embodiments, the fluid collection device may comprise an absorbent insert 320. The absorbent insert 320 may be made of a substrate, such as an absorbent or a superabsorbent material. The absorbent material may be in sheet, powdered, granular, or other form in the dry state. The absorbent material may be, for example, any hydrophilic polymer, or any porous, fibrous, synthetic, or non-synthetic material. The absorbent material may comprise a gel or polymer. For example, the absorbent material may be a synthetic or a naturally occurring polymer. The absorbent material may generally expand as liquid is absorbed. The absorbent material may form a gel with material at the target site. In some embodiments, the walls of the fluid collection chamber 310 may expand as liquid is absorbed. The absorbent insert 320 may prevent collapse of the container when a negative pressure is applied. In some embodiments, the container may be constructed of the same impermeable material that constructs the superstructure of the treatment device. This material may be embossed so that the embossed portions face inward into the container and thus provide pathways for airflow in order to enable or facilitate airflow through the fluid trap. In at least some non-limiting embodiments, the absorbent insert may uptake at least 500 g of material.

The collection device 300 may include a first tube 330 in fluid communication with the chamber of a treatment device. The collection device 300 may further include a second conduit 340 in fluid communication with a source of suction. In at least some embodiments described herein, a source of suction is capable of generating and/or maintaining sub-atmospheric pressure within an enclosed space. The device may include an inline pump. A one-way valve between the treatment device and the fluid collection device may prevent back leakage to the treatment device. A safety device, such as a transducer, may be included on the pump side so that airflow may be terminated automatically once the absorbent material is fully saturated.

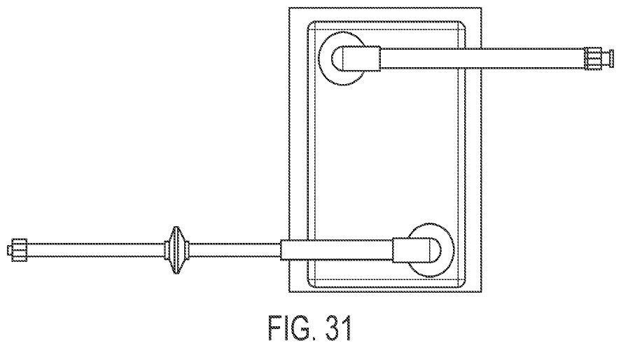

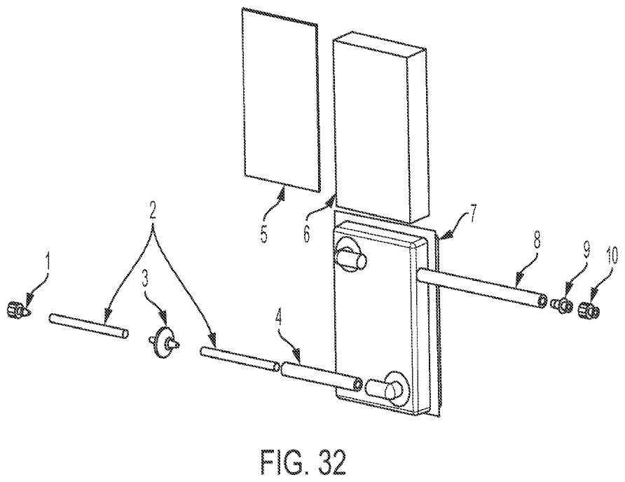

FIG. 31 is a schematic side view of a fluid collection device in accordance with one or more embodiments. It is illustrated that a piece of material can be folded and sealed on the remaining three sides around the perimeter to form the collection device. FIG. 32 is an exploded schematic side view of a fluid collection device in accordance with one or more embodiments. FIG. 32 illustrates that compartment 7 may include both a cotton material as well as a laminate absorbent sheet. Check valve (one-way) 10 prevents back flow to the treatment device and transducer 3 serves as a safety measure as described above.

In some embodiments, the fluid collection device is disposable and complies with industry waste material disposal regulations. In some embodiments of the disclosure, a patient is not restricted to a source of electricity or a battery pack. The collection device can be used with ease, so that the patient's mobility is not otherwise compromised. Further, the suction pump preferably has built-in safety limitations on force of suction, duration of operation, and overfilling of the fluid collection device.

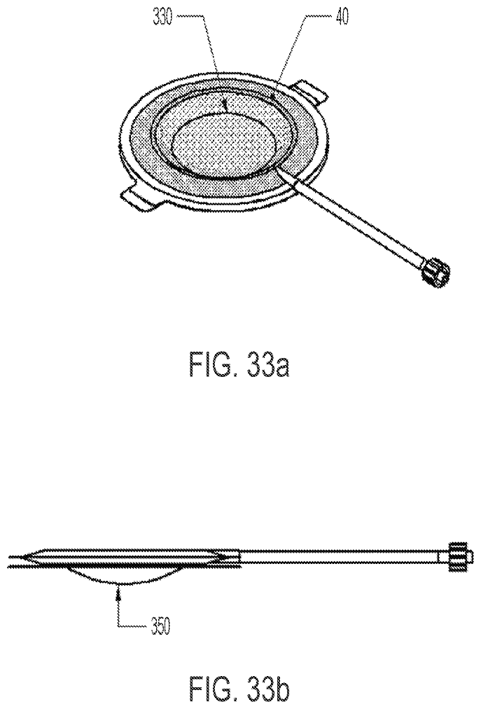

In some embodiments, the treatment device chamber may generally involve a degree of convexity or concavity to enable it to be pulled down, for example, into contact with a deep or full thickness wound, such as upon application of negative pressure. FIG. 33a illustrates the concavity of a treatment device chamber. The treatment device chamber, concave with respect to the device as applied, has a target site periphery area 330, which is placed over the periphery of the target site. A plurality of surface patterns 40 interface with the target site surface during use of the device. The plurality of surface patterns may directly contact the target site surface. The concavity of the treatment device chamber may be desirable in certain cases such as when the target site is a deep wound, for example a puncture. For example, in a deep wound, the underside of the device may be pulled into the deep parts of the wound, in order to provide negative pressure therapy and micromechanical forces to the entire deep wound surface. As shown in FIG. 33b, a treatment device chamber has a concave superstructure 350. The concavity of the superstructure of the device may provide folds that extend into the deep part of the target site to create channels for air and fluid flow.

FIGS. 36a and 36b show a treatment device 20 for delivering negative pressure, and optionally therapeutic substances, having the form of two breast shaped chambers 22 that are sized to sit on the breasts of a subject and define a treatment space 24 therein. As shown in FIG. 36b, each chamber 22 is sized and shaped to closely match the breast of the subject and has a sealing portion that secures the chamber to the skin around and underneath the breast. The dots in FIGS. 36a and 36b correspond to a plurality of surface patterns 40 that can directly contact the tissue of the breast as described herein. Each chamber has a conduit 32 that provides fluid access to the treatment space 24 of each chamber 22. As shown in FIG. 36a, the second end of each conduit 32 is connected to a Y-shaped fitting 33 that merges the independent fluid paths into one path for connection to a fluid container and a suction source 34.

In accordance with one or more embodiments, a kit for treatment may include a treatment device, a formulated therapeutic agent, and/or a fluid collection device as described herein. The kit may further include instructions to use one or more of the components for treatment.

In accordance with another aspect, there is provided a method of reducing edema at a target site on a subject. The method may comprise securing a treatment device as described herein to a periphery of the target site. The method may further comprise applying a negative pressure to the treatment space of the treatment device to allow a plurality of surface patterns on an inner surface of the treatment device to directly contact the target site and create pathways configured to evenly distribute the negative pressure in the treatment space. The method may additionally comprise maintaining the negative pressure in the treatment space for a duration of time sufficient to reduce edema of the target site. In some embodiments, the method may include reducing edema in a tissue surrounding the wound site, e.g., healthy tissue, such as where the treatment device is secured.

In some embodiments, the treatment device includes a chamber defining a treatment space, the chamber being fabricated from a substantially impermeable material and configured to conform to the shape of the periphery, an inner surface of the chamber including a plurality of surface patterns, the chamber having a sealing portion at a base, and at least one conduit connected to the chamber and in fluid communication with the treatment space so as to enable the application of a negative pressure to the treatment space. The target site may be a wound site, and can be associated with a breast, lower leg, e.g., a foot, or arm, e.g., a hand, of a subject. When associated with a breast of subject, the device may be sized and shaped to be placed on at least one breast and the surface patterns are shaped to contour to the at least one breast of the subject. In some cases, the treatment device may be secured to both breasts of the subject.

The target site may be the result of an elective procedure, such as a cosmetic procedure, or may result from a surgical procedure that produces an incision. In some cases, the target site may not be associated with any procedure and may be an area on the body experiencing edema, such as, a traumatic injury or swelling from diabetes or pregnancy. In some non-limiting embodiments, the edema management device may be placed on a wound site resulting from a surgical procedure, such as a sternotomy, thoracotomy, or the donor site of a full-thickness skin graft.

In some cases, the target site may be debrided or flushed with a sterile solution prior to or after the treatment device is secured. The exudate from the target site, including debris or drainage, may be collected and disposed of. In some cases, a therapeutic agent as described herein may be applied to the target site prior to or after the treatment device is secured.

### EXAMPLES

The function and advantage of these and other embodiments of the materials and methods disclosed herein will be more fully understood from the examples below. The following examples are intended to illustrate the benefits of the disclosed materials and methods, but do not exemplify the full scope thereof.

### Example 1

A swine study was conducted. Up to 14 partial-thickness burns were created on the dorsum of each pig. Subsequently, the burn wounds were infected with S. aureus, P. aeruginosa, A. baumannii, or C. albicans and then treated individually for 7 days with 1000×MIC of topical vancomycin, gentamicin, minocycline or 10×MIC of topical DIFLUCAN®, respectively, formulated in a 0.625% alginate hydrogel. A treatment device as described herein was used in conjunction with the antibiotic treatment. Silver sulfadiazine cream (SILVADENE®), blank 0.625% alginate hydrogel and IV antibiotics were used as controls for each topical antibiotic hydrogel. On day 7, immediately after euthanasia 6 mm wound tissue punch biopsies were collected from each wound and flash frozen for quantitative bacteriology analysis.

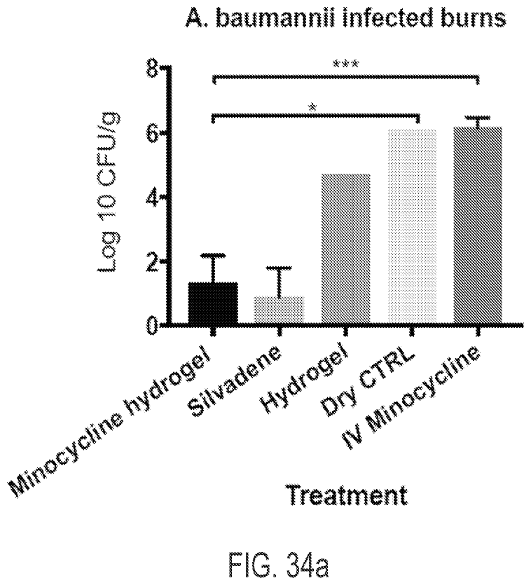

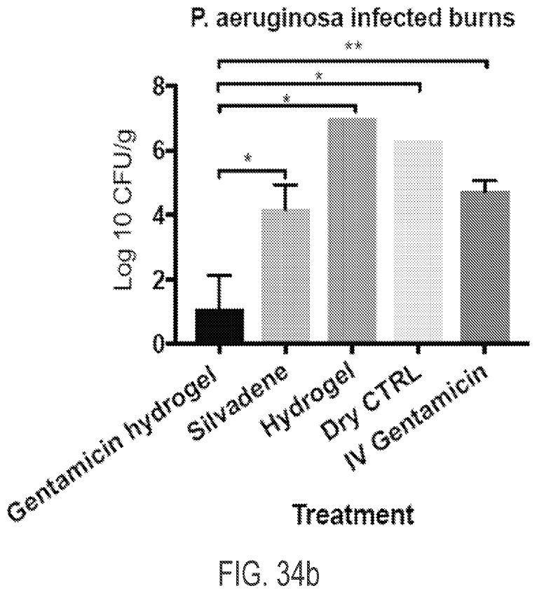

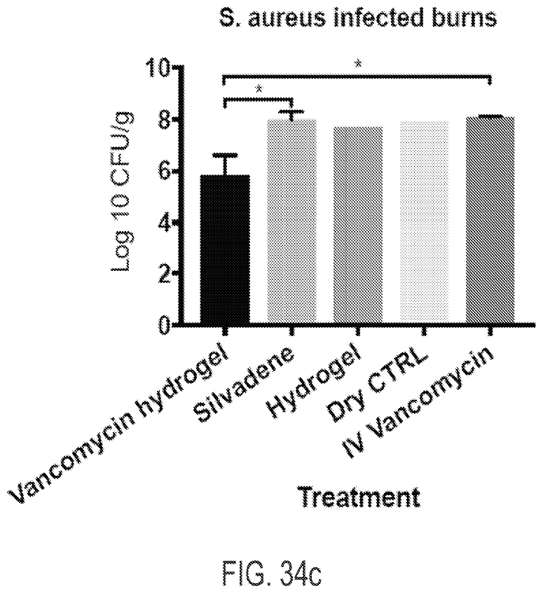

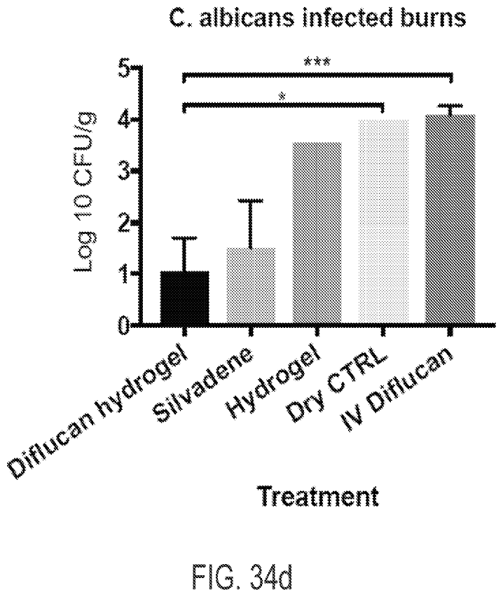

According to FIG. 34a, the results from the burns infected with A. baumannii showed that topical treatment with 1000×MIC minocycline hydrogel reduced bacterial counts more efficiently than IV minocycline and dry control (p<0.001 and p<0.01 respectively). According to FIG. 34b, in P. aeruginosa infected burns 1000×MIC gentamicin hydrogel reduced bacterial counts more efficiently than SILVADENE® cream (p<0.01), blank hydrogel (p<0.01), dry control (p<0.01) and IV minocycline. According to FIG. 34c, in S. aureus infected wounds, 1000×MIC minocycline hydrogel reduced bacterial counts in the burnt tissue more efficiently than SILVADENE® cream (p<0.01) and IV minocycline (p<0.01). According to FIG. 34d, in C. albicans infected wounds 10×MIC DIFLUCAN® hydrogel reduced bacterial counts in the burnt tissue more efficiently than dry control (p<0.01) and IV DIFLUCAN® (p<0.01).

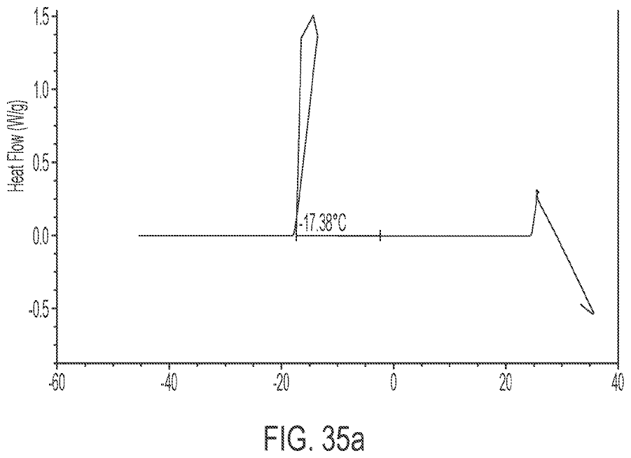

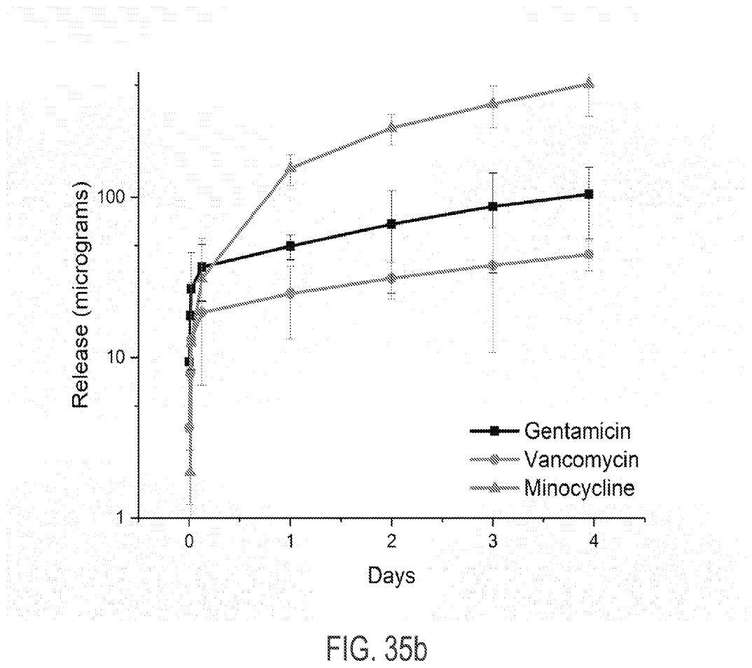

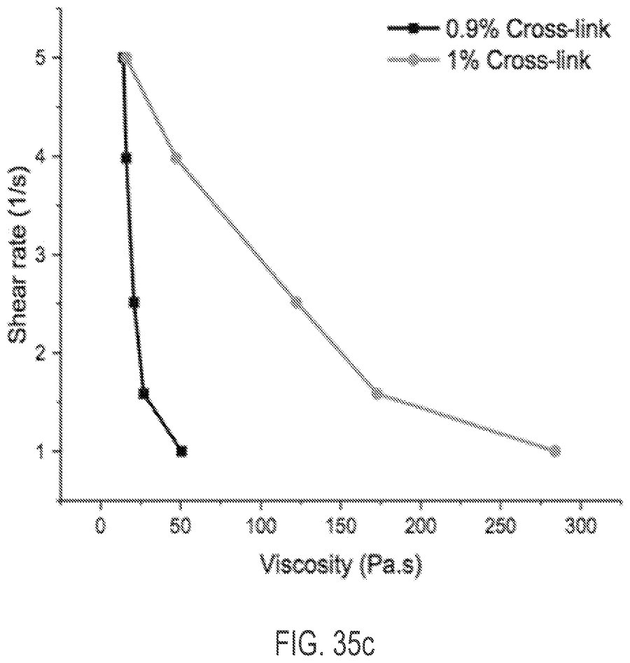

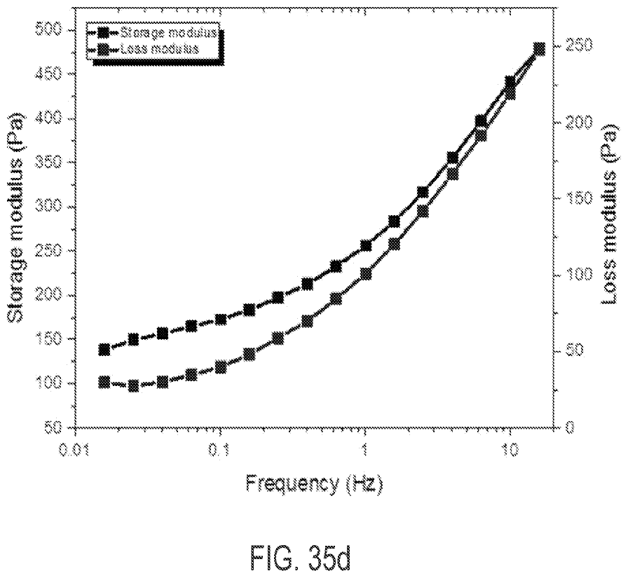

Thermal stability data pertaining to the 0.625% alginate hydrogel is presented in FIG. 35a illustrating Differential Scanning Calorimetry (DSC) measurements of the hydrogel sample from room temperature to −45° C. at a rate of 0.1° C. per minute. The freezing point is indicated by a positive heat flow into the hydrogel sample during the cooling cycle beginning at −17.38° C.

Cumulative drug release data pertaining to the 0.625% alginate hydrogel is presented in FIG. 35b. Hydrogel samples of 500 μL were placed in Trans-well membrane plates, with 500 μL of PBS in the bottom well. At each time point, 100 μL aliquots were taken from the bottom well, while 100 μL of fresh PBS was added simultaneously. Aliquots were analyzed using Liquid chromatography-mass spectrometry (LCMS) to quantify the concentrations of the respective antibiotics.

Rheology data pertaining to the 0.625% alginate hydrogel is presented in FIG. 35c. On a stress-controlled rheometer, homogenized hydrogels were analyzed via shear rate sweeps to calculate the viscosity, as an indication of injectability. The geometry used was rough-surfaced 1° 40 mm Cone-and-Plate at a gap of 400 μm.

Storage modulus and loss modulus data pertaining to the 0.625% alginate hydrogel is presented in FIG. 35d. The storage modulus is larger than the loss modulus, indicative of a gel, and not a viscous liquid. Also, the moduli are relatively low, demonstrating that it is a very soft hydrogel. A strain sweep is used to determine the linear-viscoelastic area of the hydrogel, and a frequency sweep is used to determine the linear equilibrium modulus plateau of the hydrogel.

### Example 2

In this example, a treatment device as described herein was utilized on closed incision surgical wounds from differing procedures on a large patient population to investigate in a clinical setting the efficacy of the treatment device.

### Subject Enrollment

Potential subjects were screened over 5 months at a single center as part of this prospective case series study. Inclusion criteria included any patient 18 to 85 years of age scheduled to undergo a surgical intervention for which an incision would be made and primarily closed at the time of operation. Exclusion criteria included patients with active infection, use of immunosuppressive agents, radiation or chemotherapy within the past 30 days, pregnancy, or inability to give informed consent. Comorbidities screened for included hypertension, diabetes, coronary artery disease, history of smoking, and active malignancy, among others. All patients enrolled in the study gave written informed consent prior to enrollment.

### Negative-Pressure Platform Wound Dressing (NP-PWD)

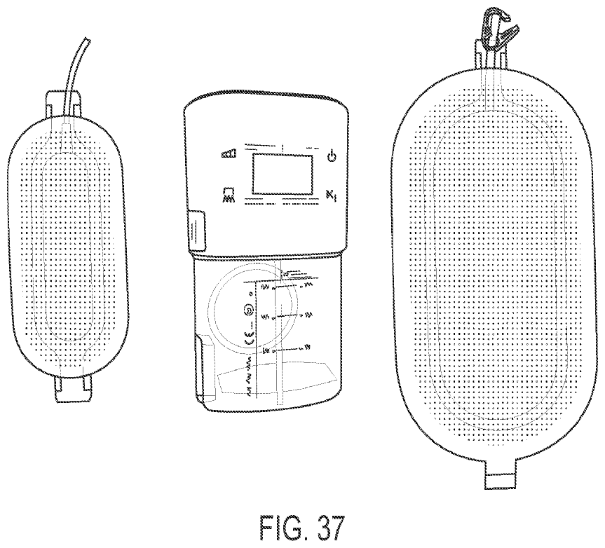

NP-PWDs (Applied Tissue Technologies LLC, Hingham, Mass.) were used with the INVIA® Motion NPWT pump (Medela, Baar, Switzerland) at −80 mm Hg continuous pressure in the study. A negative pressure tube was connected to each PWD and to the negative pressure pump. Oblong NP-PWD with wound openings measuring 1″×3″ (Part no. AT1073-01) and 3″×5″ (Part no. AT1074-01) were utilized in this study (FIG. 37).

### Study Design

Immediately following incision closure in the operating room, the length of the incision was measured, and photographs obtained. The NP-PWD was applied using sterile techniques. Excess hair adjacent to the wound was removed with a razor prior to application. The backing on the underside of the NP-PWD was then removed and the dressing was gradually applied so that the center of the NP-PWD was directly over the center of the wound and the adhesive in contact with intact skin surrounding the wound. The INVIA® motion pump, collection canister, and tubing were then assembled, and the pump securely connected to the NP-PWD. The pump was then powered on and negative pressure set to −80 mm Hg. The NP-PWD was removed at the first post-operative check (POC) between postoperative days (PODs) 3-5. Patients were required to undergo an additional POC between PODs 9-14.

### Incision Assessment

POCs consisted of incision assessment, measurement, photography, and adverse event (AE) screening. Assessments specifically addressed erythema, drainage, itching, and pain at the incision site. At each POC, two members of the study team indicated presence or absence of the above physical exam findings. The same study members assessed the incisions at each time point to maintain consistency. Evaluation of erythema and drainage were made qualitatively by members of the study team while assessment of itching and pain were made based on patient reports. Incision measurements were recorded in centimeters at each POC by members of the study team. A digital photograph of each wound was obtained at each POC as well. All AEs were recorded and assessed for relation to the NP-PWD.

### Results

No difficulties directly related to patients' willingness to participate in the study were identified. All patients enrolled in the study maintained the dressing for the duration intended. All patients enrolled in the study were present at both of their scheduled POCs and no patients were lost to follow-up.

There was no difficulty applying the dressing. As there are no adjustments to size or shape that need to be made to the dressing prior to application, those applying the dressing felt comfortable doing so after 1-2 applications.

A total of eight non-consecutive patients with ten incisions were included in the study. Five patients were male. Three patients were female. Half of the patients enrolled were Hispanic (4, 50%) and half were Caucasian (4, 50%). The median age was 56 years (IQR 53-74 years) and median BMI was 28.4 (IQR 25.2-35) (Table 2).

TABLE 2
Overview of Demographic Data for Study Population

| Demographics | | |
|---|---|---|
| Total number enrolled, n | 8 | |
| Age (years), median (IQR) | 56 | (53.5,74) |
| Sex, n (%) | | |
| Male | 5 | (62.5) |
| Female | 3 | (37.5) |
| Ethnicity, n (%) | | |
| Caucasian | 4 | (50) |
| Hispanic | 4 | (50) |
| BMI, median (IQR) | 28.4 | (25.2, 35) |

IQR: Interquartile range,
BMI: Body mass index

### Comorbidities

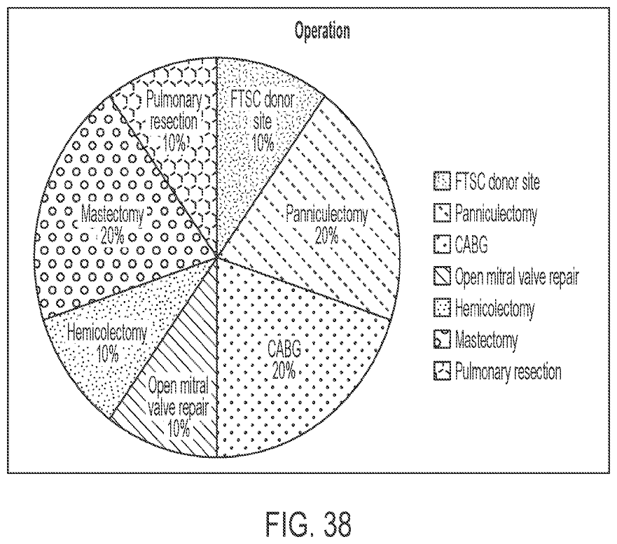

All patients had at least one comorbidity recorded. The most common comorbidities present were hypertension (n=6), active malignancy (n=4), and type 2 diabetes mellitus (n=2). All comorbidities are listed in Table 3. Operations ranged from full-thickness skin graft (FTSG) donor site obtained from the groin (n=1), panniculectomy (n=2), coronary artery bypass graft (CABG, n=2), open mitral valve repair (n=1), hemicolectomy (n=1), mastectomy (n=2), and pulmonary resection (n=1) as illustrated in FIG. 38.

TABLE 3
All Comorbidities of Study Subjects

| Comorbidities | n (%) | |
|---|---|---|
| Hypertension | 6 | (75) |
| Active malignancy | 4 | (50) |
| Type 2 diabetes mellitus | 2 | (25) |
| Neuropathy | 2 | (25) |
| Coronary artery disease | 2 | (25) |
| GERD | 2 | (25) |
| Asthma | 1 | (12.5) |
| Chronic kidney disease | 1 | (12.5) |
| Smoking | 1 | (12.5) |
| Hyperlipidemia | 1 | (12.5) |
| Atrial fibrillation | 1 | (12.5) |
| Mitral valve stenosis | 1 | (12.5) |

GERD: Gastroesophageal reflux disease

### Incision Length and Dressing Duration

Median initial incision length was 20 cm (IQR 7.4 cm-20 cm). Incision length remained the same throughout the duration of the treatment. For the majority of incisions, the dressing remained in place for 3 days (60%). All dressings were removed by POD 5 (Table 4).

TABLE 4
Incision Length and Negative Pressure Wound Therapy Duration

| Incision | | Incision Length | Duration |
|---|---|---|---|
| Number | Operation | (cm) | (Days) |
| 1 | FTSG donor site | 8 | 4 |
| 2 | Panniculectomy | 20 | 3 |
| 3 | Panniculectomy | 20 | 3 |
| 4 | CABG | 20 | 3 |
| 5 | CABG | 20 | 3 |
| 6 | Open mitral valve | 23 | 4 |
| | repair | | |
| 7 | Hemicolectomy | 20 | 3 |
| 8 | Mastectomy | 7 | 5 |
| 9 | Mastectomy | 7 | 5 |
| 10 | Pulmonary resection | 7.5 | 3 |

FTSG: Full-Thickness Skin Graft;
CABG: Coronary Artery Bypass Graft

### Incision Assessment

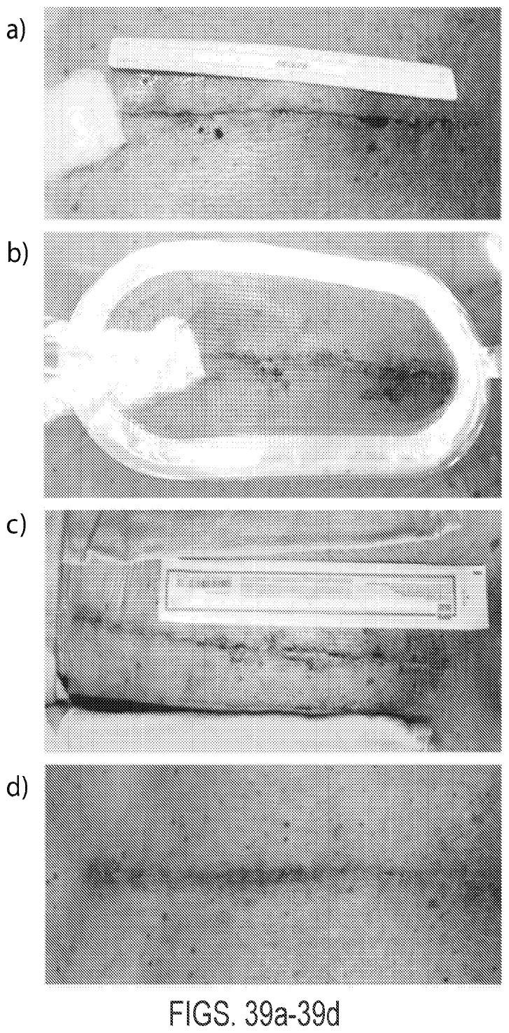

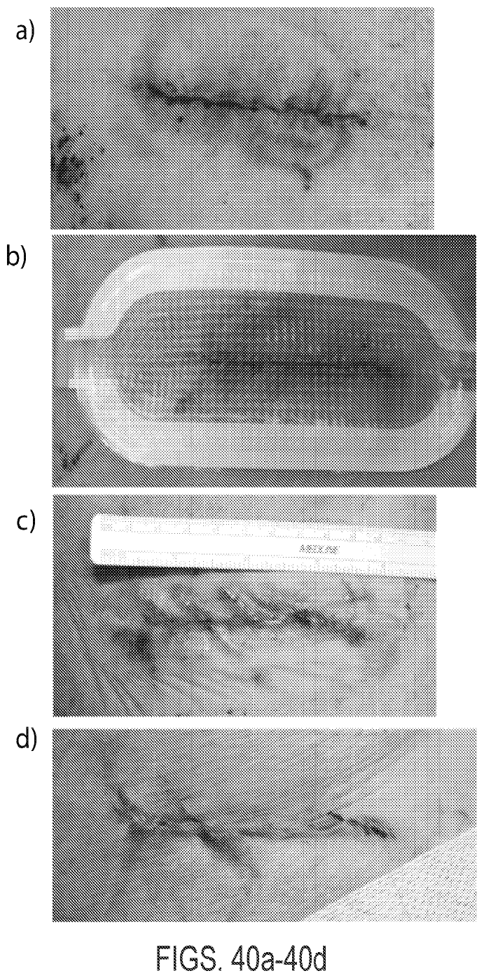

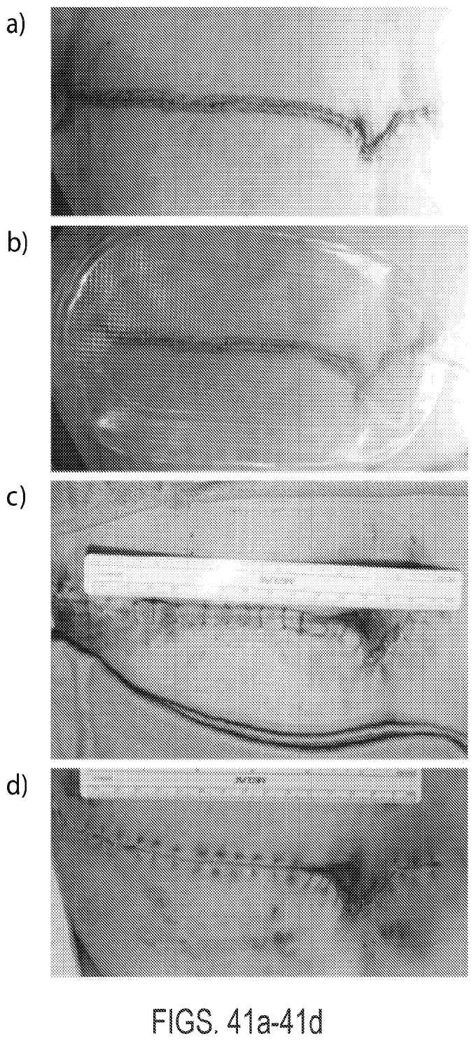

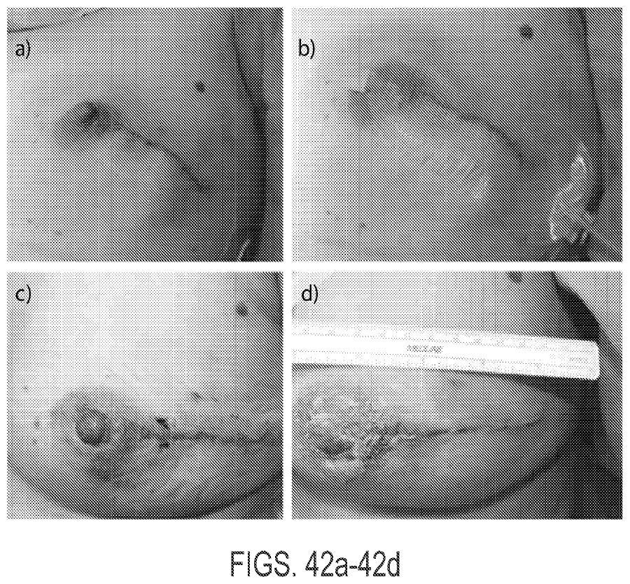

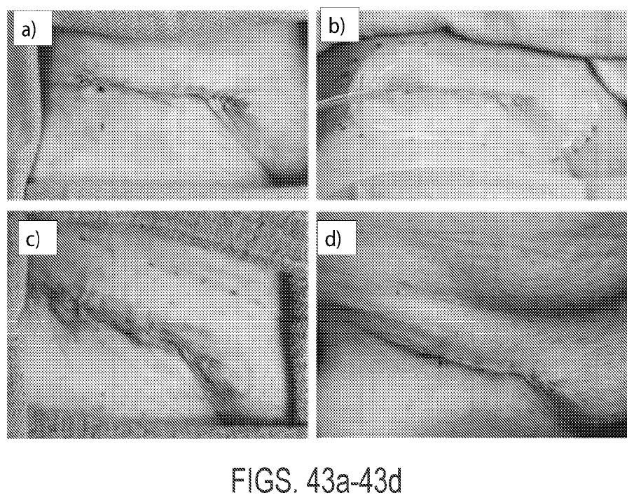

At the first POC, all incisions were observed to have serosanguinous (SS) drainage. Less than half of the incisions were erythematous (40%) and only 2 were reported to be painful (20%). By the second POC, all 55 drainage had resolved. Three incisions were found to be erythematous (30%), 1 was reported to be itchy (10%), and none were painful (Table 5, FIGS. 39a-39d, 40a-40d, 41a-41d, 42a-42d, and 43a-43d). No wound breakdown or infection was observed. There were three adverse events: small blisters were noted under the adhesive of two incisions, and for one patient, the tubing was disconnected upon transfer from the ICU resulting in interruption of therapy.

TABLE 5
Post-Operative Check Assessments

| | | |
|---|---|---|
| Incision Assessment | POD 3-6 | POD 9-14 |

| | | | | |
|---|---|---|---|---|
| Erythema, n (%) | 4 | (40) | 3 | (30) |
| Drainage, n (%) | 10 | (100) | 0 | (0) |
| Itching, n (%) | 0 | (0) | 1 | (10) |
| Pain, n (%) | 2 | (20) | 0 | (0) |

### DISCUSSION

This study examined the use of the NP-PWD in a clinical setting. In this study, the dressing was applied onto a variety of closed surgical incisions following operations to different areas of the body including the breast, chest or chest walls, abdomen, and groin. No wound infection occurred during this study. Of note, the patients included in this study had significant comorbidities, including diabetes and active malignancies, that are known to impair wound healing. The dressing was easy to apply by the clinical staff, patients did not report any intolerable side effects, and incisions healed well without signs of infection or dehiscence. In the case of the blisters that developed under the adhesive of two incisions, this was more likely to be due to incision location (panniculectomy) and patient factors as opposed to the device itself.

The NP-PWD devices disclosed herein remedy the deficiencies of currently available negative pressure wound care devices. For example, in currently available devices, the time to cut the foam to the shape of the wound is significant. The opacity of the foam within current devices prevents assessments while the devices are in place and can serve as a nidus for infection or interrupt the formation of healthy granulation tissue upon removal. Current negative pressure wound care devices require relatively high negative pressure settings to reach therapeutic levels of negative pressure on the wound bed. High negative pressure levels can result in discomfort for the patient and even lead to ischemia of wounds that are already poorly vascularized. In addition, currently available negative pressure wound care devices are, in some circumstances, not placed directly onto the incision, but rather on top of another dressing making the process more time consuming and more complicated with a steeper learning curve for providers. The NP-PWD devices disclosed herein addresses those limitations by providing an easy-to-apply, transparent, foam-less dressing that can be placed in the OR and removed within days after incision creation.

The NP-PWD devices disclosed herein provide a novel closed incision negative pressure therapy (NPT) that provides an easy-to-apply, transparent, foam-less dressing that can be placed in the OR and removed within days after incision creation.

The strengths of this study include that the study design proved to be reasonable both in terms of patient and provider participation, and no significant concerns were identified regarding the dressing within this case series, such as poor wound healing, infection, or device intolerance by enrolled patients. Incisions healed well over the course of the study with the majority being free of drainage, erythema, itching, and pain by 14 days postoperatively. It is noted that, as for all single-institution studies, the results are only representative of the population at one institution and so further multi-institutional studies must be performed in order to generalize the results. The small number of patients included in the study and the majority of data collected being qualitative are also to be considered for future studies. Selection bias was present during this study as only patients with planned surgical interventions were included. It is recognized that measurement bias and lack of control are inherent limitations of case series that were present within this study. Overall, this case series provided preliminary data based on which additional, more rigorous studies evaluating the use of NP-PWD on closed incisions to improve wound healing can be developed.

### Prophetic Example 1

The goal of this study will be to determine the efficacy of using a treatment device in conjunction with formulated high-dose therapeutic agents as described herein to ease pain and reduce or eradicate infection as compared to the standard of care dressing in human patients.

Pre-clinical experiments in swine have demonstrated that immediate treatment with the present wound treatment devices stop wound depth progression and prevent infection.

In this study, a large but safe dose (1000×MIC) of an antibiotic and analgesic in a gel will be used to immediately treat patients with traumatic extremity wounds and burns (total body surface area of 5 to 30%). There will be 25 standard of care (control) devices and 25 wound treatment devices in accordance with one or more present embodiments used in the study. The devices will be left in place for at least 48 and up to 96 hours at which time it will be removed and the wound cultured and debrided. The antibiotic and analgesic concentrations in the gel/fluid within the devices as well in the serum will be measured every 24 hours.

Quantitative bacterial counts (CFU) will demonstrate significant reduction or eradication of infection in connection with the devices and formulations described herein in comparison to the standard of care. Likewise, pain as assessed by tonometry and a pain analog scale will demonstrate reduction or even elimination of pain compared to the standard of care.

It is to be appreciated that embodiments of the methods, devices, and apparatuses discussed herein are not limited in application to the details of construction and the arrangement of components set forth in the above description or illustrated in the accompanying drawings. The methods, devices, and apparatuses are capable of implementation in other embodiments and of being practiced or of being carried out in various ways. Examples of specific implementations are provided herein for illustrative purposes only and are not intended to be limiting. In particular, acts, elements and features discussed in connection with any one or more embodiments are not intended to be excluded from a similar role in any other embodiment.

Also, the phraseology and terminology used herein is for the purpose of description and should not be regarded as limiting. Any references to embodiments or elements or acts of the systems and methods herein referred to in the singular may also embrace embodiments including a plurality of these elements, and any references in plural to any embodiment or element or act herein may also embrace embodiments including only a single element. The use herein of “including,” “comprising,” “having,” “containing,” “involving,” and variations thereof is meant to encompass the items listed thereafter and equivalents thereof as well as additional items. References to “or” may be construed as inclusive so that any terms described using “or” may indicate any of a single, more than one, and all of the described terms. Any references to front and back, left and right, top and bottom, upper and lower, and vertical and horizontal are intended for convenience of description, not to limit the present devices and methods or their components to any one positional or spatial orientation.

Having described above several aspects of at least one embodiment, it is to be appreciated that various alterations, modifications, and improvements will readily occur to those skilled in the art. Such alterations, modifications, and improvements are intended to be part of this disclosure and are intended to be within the scope of the invention. Accordingly, the foregoing description and drawings are by way of example only.

What is claimed is:

1. A method of reducing edema at a target site on a subject, comprising:
securing a treatment device to a periphery of the target site, the treatment device comprising:
a chamber defining a treatment space, the chamber being fabricated from a substantially impermeable material and configured to conform to the shape of the periphery, an inner surface of the chamber including a plurality of surface patterns, the chamber having a sealing portion at a base; and
at least one conduit connected to the chamber and in fluid communication with the treatment space so as to enable the application of a negative pressure to the treatment space;
applying a negative pressure to the treatment space of the treatment device to allow the plurality of surface patterns to directly contact the target site and create pathways configured to evenly distribute the negative pressure in the treatment space; and
maintaining the negative pressure in the treatment space for a duration of time sufficient to reduce edema of the target site.

2. The method of claim 1, wherein the target site comprises a wound site.

3. The method of claim 2, wherein the treatment device reduces edema in a tissue surrounding the wound site.

4. The method of claim 1, wherein the target site is associated with a breast of the subject.

5. The method of claim 4, wherein the treatment device is sized and shaped to be placed on at least one breast of the subject.

6. The method of claim 5, wherein the plurality of surface patterns are shaped to contour to the at least one breast of the subject.

7. The method of any one of claims 4-6, wherein a treatment device is secured to both breasts of the subject.

8. The method of claim 1, wherein the plurality of surface patterns comprises a plurality of embossed structures.

9. The method of claim 1, wherein the target site is associated with a lower leg of the subject.

10. The method of claim 9, wherein the target site is associated with a foot of the subject.

11. The method of claim 1, wherein the target site is associated with an arm of the subject.

12. The method of claim 11, wherein the target site is associated with a hand of the subject.

13. The method of claim 1, wherein the target site is associated with a chest of the subject.

14. The method of claim 1, wherein the target site is associated with an abdomen of the subject.

15. The method of claim 1, wherein the target site results from an elective procedure.

16. The method of claim 15, wherein the elective procedure is a cosmetic procedure.

17. The method of claim 1, wherein the target site results from a skin graft procedure.

18. The method of claim 1, wherein the target site results from a surgical procedure.

19. The method of claim 18, wherein the surgical procedure produces an incision.

20. The method of claim 1, further comprising collecting exudate from the target site.

21. The method of claim 1, further comprising debriding the target site prior to the securing of the treatment device.

22. The method of claim 1, further comprising flushing the target site with a sterile solution.

23. The method of claim 1, further comprising applying a therapeutic agent to the target site.

24. An edema management device for reducing edema at a target site on a subject, comprising:
a chamber defining a treatment space, the chamber being fabricated from a substantially impermeable material and configured to conform to a shape of a periphery of the edemic target site, an inner surface of the chamber including a plurality of surface patterns;
a sealing portion at a base of the chamber configured to be secured to the skin of the periphery around the edemic target site, the sealing portion comprising an adhesive; and
at least one conduit having a first end and a second end, the first end connected to the chamber and in fluid communication with the treatment space so as to enable applying a negative pressure to the treatment space.

25. The device of claim 24, wherein the chamber is constructed and configured to enclose a target site on the subject experiencing edema.

26. The device of claim 24, wherein the at least one conduit is configured to remove fluid from, or introduce fluid to, the treatment space.

27. The device of claim 24, wherein the plurality of surface patterns are shaped to contact and contour to the target site.

28. The device of claim 24, wherein the plurality of surface patterns are positioned at a distance of about 0.2 mm to about 10 mm apart from one another.

29. The device of claim 24, wherein the plurality of surface patterns have a height of about 0.1 mm to about 5 mm.

30. The device of claim 24, wherein the plurality of surface patterns is positioned in a uniform pattern on the inner surface of each chamber.

31. The device of claim 24, wherein the plurality of surface patterns is positioned in a non-uniform pattern on the inner surface of each chamber.

32. The device of claim 24, wherein each of the plurality of surface patterns has a shape selected from the group consisting of a cone, a pyramid, a pentagon, a hexagon, a half sphere, a dome, a rod, an elongated ridge with round sides, and an elongated ridge with square sides.

33. The device of claim 24, wherein the plurality of surface patterns intrudes into the treatment space in a direction generally perpendicular to the inner surface of each chamber.

34. The device of claim 24, wherein the plurality of surface patterns comprises a plurality of embossed structures.

35. The device of claim 24, wherein the chamber does not include a porous insert.

36. The device of claim 24, further comprising a suction device for applying the negative pressure, a fluid trap, and an exhaust port, wherein the suction device is in fluid communication with the treatment space of each chamber, the fluid trap and the exhaust port, and wherein the fluid trap is fluidly positioned between the suction device and the exhaust port.

37. The device of claim 24, wherein the target site is associated with a breast of the subject.

38. The device of claim 24, wherein the target site is associated with a lower leg of the subject.

39. The device of claim 38, wherein the target site is associated with a foot of the subject.

40. The device of claim 24, wherein the target site is associated with an arm of the subject.

41. The device of claim 40, wherein the target site is associated with a hand of the subject.

42. A edema management device for at least one breast of a subject, comprising,
a chamber having an inner surface defining a treatment space sized and shaped to enclose at least one breast, the inner surface comprising a plurality of surface patterns configured to directly contact the at least one breast and to create pathways for distributing a negative pressure between the inner surface and the at least one breast;
a sealing portion at a base of the chamber configured to be secured to the skin of the chest wall around the at least one breast, the sealing portion comprising an adhesive; and
at least one conduit having a first end and a second end, the first end connected to the chamber and in fluid communication with the treatment space so as to enable applying a negative pressure to the treatment space.

43. The device of claim 42, wherein the chamber is sized and shaped to enclose both breasts of the subject.

44. The device of claim 42, wherein the chamber is configured to provide support to the at least one breast when a negative pressure is applied.

45. The device of claim 42, wherein the chamber is associated with an undergarment configured to provide support to the at least one breast.

46. The device of claim 44, wherein the undergarment comprises an outer supporting structure enclosing the chamber.

47. The device of claim 46, wherein the outer supporting structure is a fabric material.

48. The device of claim 42, wherein the chamber is fabricated from a substantially impermeable material.

49. The device of claim 42, wherein the at least one conduit is configured to remove fluid from, or introduce fluid to, the treatment space.

50. The device of claim 42, wherein the plurality of surface patterns are shaped to contour to the at least one breast.

51. The device of claim 42, wherein the plurality of surface patterns are positioned at a distance of about 0.2 mm to about 10 mm apart from one another.

52. The device of claim 42, wherein the plurality of surface patterns have a height of about 0.1 mm to about 5 mm.

53. The device of claim 42, wherein the plurality of surface patterns is positioned in a uniform pattern on the inner surface of each chamber.

54. The device of claim 42, wherein the plurality of surface patterns is positioned in a non-uniform pattern on the inner surface of each chamber.

55. The device of claim 42, wherein each of the plurality of surface patterns has a shape selected from the group consisting of a cone, a pyramid, a pentagon, a hexagon, a half sphere, a dome, a rod, an elongated ridge with round sides, and an elongated ridge with square sides.

56. The device of claim 42, wherein the plurality of surface patterns intrudes into the treatment space in a direction generally perpendicular to the inner surface of each chamber.

57. The device of claim 42, wherein the chamber does not include a porous insert.

58. The device of claim 42, further comprising a suction device for applying the negative pressure, a fluid trap, and an exhaust port, wherein the suction device is in fluid communication with the treatment space of each chamber, the fluid trap and the exhaust port, and wherein the fluid trap is fluidly positioned between the suction device and the exhaust port.
